(19) United States
(12) Patent Application Publication
(10) Pub. No.: US 2026/0341518 A1
(43) Pub. Date: Oct. 29, 2026

(54) METHODS AND SYSTEMS FOR AI-BASED IMMUNE PROFILING FOR CANCER PATIENT STRATIFICATION

(71) Applicant: JANSSEN RESEARCH & DEVELOPMENT, LLC, Raritan, NJ (US)

(72) Inventors: Albert Juan RAMON, Raritan, NJ (US); Kristopher STANDISH, Raritan, NJ (US); Chaitanya PARMAR, Raritan, NJ (US); Brendon LUTNICK, Raritan, NJ (US); Stephen YIP, Raritan, NJ (US); Brandon GINLEY, Raritan, NJ (US)

(73) Assignee: JANSSEN RESEARCH & DEVELOPMENT, LLC, Raritan, NJ (US)

(21) Appl. No.: 19/106,290

(22) PCT Filed: Sep. 1, 2023
(86) PCT No.: PCT/IB2023/058673

§ 371 Date: Feb. 25, 2025

Related U.S. Application Data
(60) Provisional application No. 63/403,652, filed on Sep. 2, 2022.

(51) Int. Cl. G16H 50/30; G16B 25/10; G16B 40/20; G16H 10/40; G16H 30/20

(52) U.S. Cl. CPC G16H 50/30; G16B 25/10; G16B 40/20; G16H 10/40; G16H 30/20

(57) ABSTRACT

Systems and methods for predicting patient response to a therapeutic are disclosed. The systems and methods may include a data store for storing an image of a tissue sample from a patient, the image comprising one or more features of the tissue sample; a computing comprising: a first neural network model configured to generate one or more programmed death-ligand 1 (PD-L1) expression level indicators or configured to generate one or more programmed death-1 (PD-1) expression level indicators; a second neural network model configured to calculate one or more tumor infiltrating lymphocyte (TIL) related metrics; and/or a third neural network model configured to predict a response status of the patient to the immuno-oncology therapeutic in accordance with the one or more PD-L1 expression level indicators and/or one or more PD-1 expression level indicators, respectively, and/or the one or more TIL-related metrics; and a display system configured to display a result that indicates the response status of the patient.

This application is a National Stage Application from PCT/IB2023/058673, filed Sep. 1, 2023, which claims priority to U.S. Provisional Patent Application Ser. No. 63/403,652, filed Sep. 2, 2022, which is incorporated by reference herein in its entirety.

### BACKGROUND

Cancer immunotherapy is an emerging discipline of cancer treatments whose main goal is to harness the patient's own immune system to recognize and destroy tumor cells. Various forms of immunotherapy are being developed and are in variable stages of preclinical and clinical development. Forms of immunotherapy include, but are not limited to, monoclonal antibodies and adoptive cell transfer.

Checkpoint inhibition is one aspect of cancer immunotherapy and shows efficacy in both clinical and preclinical studies as an adjuvant and alternative to traditional cancer therapies. The effectiveness of checkpoint inhibition results from releasing T cells from the inhibitory effects of checkpoint molecules. T cells in the tumor microenvironment (TME), in response to multiple TME-derived factors, increase expression of checkpoint molecules, such as programmed cell death 1 (PD-1) and programmed cell death 1 ligand 1 (PD-L1).

Cancer therapeutics including checkpoint inhibitors (including monoclonal antibodies) provide challenges when the therapeutics lack efficacy for a patient, either with primary resistance or acquired resistance. Primary resistance can occur when a cancer does not respond to an immunotherapeutic strategy, and in the case of checkpoint inhibitors can occur when the cancer cells fail to express them, thereby providing no target for the therapeutic.

### SUMMARY

Embodiments of the disclosure include a system for predicting patient response to an immuno-oncology therapeutic, the system comprising: a data store for storing an image of a tissue sample from a patient, the image comprising one or more features of the tissue sample; a computing device communicatively connected to the data store, the computing device comprising: a first neural network model configured to generate one or more tumor mutation burden (TMB) indicators from the one or more features; a second neural network model configured to generate one or more programmed death-ligand 1 (PD-L1) expression level indicators from the one or more features and/or configured to generate one or more programmed death-1 (PD-1) expression level indicators from the one or more features; a third neural network model configured to calculate one or more tumor infiltrating lymphocyte (TIL)-related metrics from the one or more features; or a fourth neural network model configured to predict a response status of the patient to the immuno-oncology therapeutic in accordance with the one or more TMB indicators, the one or more PD-L1 expression level indicators and/or the one or more PD-1 expression level indicators, and/or the one or more TIL-related metrics; and a display system communicatively connected to the computing device and configured to display a result that indicates the response status of the patient.

In specific embodiments, the prediction of the response status of the patient to the immuno-oncology therapeutic is performed in accordance with one or more medical history features of the patient. The third neural network model may comprise a segmentation algorithm configured to generate an identification of a tumor region in the tissue sample based on the one or more features, wherein the identification is used to further generate the one or more TIL-related metrics. In specific embodiments, the segmentation algorithm is configured to predict a TIL mask, which is used in combination with a tumor mask to calculate one or more TIL related metrics. In particular embodiments, the one or more TIL-related metrics comprises a predicted number of TIL clusters in the identified tumor region; a predicted size of TIL clusters in the identified tumor region; a predicted TIL cluster spread in the identified tumor region; a total count of TILs in the identified tumor region; distance between TIL cells vs. distance between tumor cells; distance of TIL cells from tumor cells; average size of TIL cells; a stromal TIL % (sTIL %) (which may be calculated as a percent of stroma in the identified tumor region in the tissue sample that is infiltrated by TIL); and/or an intra-tumor TIL % (iTIL %), which may be calculated as a percent of the identified tumor region in the tissue sample that is infiltrated by TIL. In various embodiments, the first neural network model comprises a classification algorithm and the one or more TMB indicators comprises a TMB indicator for the tissue sample that is either a TMB positive classification or TMB negative classification. The classification algorithm may generate the TMB positive classification if the tissue sample has a TMB value that exceeds a pre-set TMB threshold value. In some cases, the first neural network model comprises a regression algorithm and the one or more TMB indicators comprises a TMB indicator that is a quantitative TMB score. In various embodiments, the second neural network model comprises a classification algorithm and the one or more PD-L1 expression level indicators comprises a PD-L1 expression level indicator that is either a PD-L1 expression positive classification or a PD-L1 expression level negative classification. The second neural network model may comprise a classification algorithm and the one or more PD-1 expression level indicators comprises a PD-1 expression level indicator that is either a PD-1 expression positive classification or a PD-1 expression level negative classification. In certain embodiments, the classification algorithm generates the PD-L1 expression positive classification if the tissue sample has a PD-L1 expression level value that exceeds a pre-set PD-L1 expression level value. In some embodiments, the classification algorithm generates the PD-1 expression positive classification if the tissue sample has a PD-1 expression level value that exceeds a pre-set PD-1 expression level value. The second neural network model may comprise a regression algorithm and the one or more PD-L1 expression level indicators comprises a quantitative PD-L1 score. In certain embodiments, the second neural network model comprises a regression algorithm and the one or more PD-1 expression level indicators comprises a quantitative PD-1 score. The quantitative PD-L1 score and/or the quantitative PD-1 score may be tumor proportion score (TPS). The quantitative PD-L1 score and/or the quantitative PD-1 score may be a combined positive score (CPS).

In some embodiments, the third neural network model is further configured to generate one or more TIL masks from the one or more features; and wherein the fourth neural network model is further configured to predict the response status of the patient to the immuno-oncology therapeutic using the one or more TIL masks.

In various embodiments, the immuno-oncology therapeutic is a check point inhibitor, and the check point inhibitor may be a PD-L1 inhibitor or PD-1 inhibitor. Any tissue sample may be from the patient's bladder; and in some embodiments the patient response to the immuno-oncology therapeutic is related to bladder cancer. The tissue sample may be from a patient known to have PD-L1-positive cancer. The tissue sample may be from a patient known to have PD-1-positive cancer. In specific embodiments, the tissue sample is from a patient known to have any one or more of the following: non-small cell lung cancer, melanoma, Hodgkin lymphoma, bladder cancer, kidney cancer, and breast cancer. The immuno-oncology therapeutic targets PD-L1 or PD-1, in specific embodiments. In some embodiments, the immuno-oncology therapeutic comprises one or more antibodies, one or more adoptive cell therapies, or a combination thereof. The one or more antibodies may comprise at least one of: a monoclonal antibody, a bispecific antibody, or a trispecific antibody. In some embodiments, the one or more adoptive cell therapies comprises immune cells that express one or more engineered antigen receptors. In some embodiments, the one or more engineered antigen receptors comprises at least one of: a chimeric antigen receptor, a non-natural T cell receptor, or a combination thereof. In certain embodiments, the one or more adoptive cell therapies comprises at least one of: T cells, natural killer cells, natural killer T cells, or a combination thereof.

In particular embodiments, the third neural network model comprises a region/TIL segmentation algorithm and a cell segmentation algorithm. The cell segmentation algorithm may calculate one or more cell-based features. In particular embodiments, the one or more cell-based features include morphology, color (nuclear), color (extra-nuclear), texture (energy, correlation, contrast, homogeneity, dissimilarity, or angular second moment). In some embodiments, the cell segmentation algorithm generates a number of TIL cells or average number of TIL cells. The cell segmentation algorithm may generate an average tumor cell size, an average TIL cell size, an average distance between TIL cells, an average color of TIL cells, and/or an average distance between TIL cells vs tumor cells, in certain embodiments.

Embodiments of the disclosure include methods for predicting patient response to an immuno-oncology therapeutic, the method comprising obtaining, by one or more processors, an image of a tissue sample from a patient, the image comprising one or more features of the tissue sample; generating, by the one or more processors, via a first neural network model, one or more tumor mutation burden (TMB) indicators from the one or more features; generating, by the one or more processors, via a second neural network model, one or more programmed death-ligand 1 (PD-L1) expression level indicators from the one or more features and/or one or more programmed death-1 (PD-1) expression level indicators from the one or more features; generating, by the one or more processors, via a third neural network model, one or more tumor infiltrating lymphocyte (TIL) related metrics from the one or more features; and predicting, by the one or more processors, via a fourth neural network model, a response status of the patient to the immuno-oncology therapeutic using the one or more TMB indicators, the one or more PD-L1 expression level indicators or the one or more PD-1 level indicators, and the one or more related metrics. In some embodiments, the method further comprises selecting, in accordance with the response status of the patient, whether to include the patient in a cohort of patients participating in a clinical trial. In some embodiments, the method further comprises displaying, on a display screen, a graphical representation indicating the response status of the patient.

In certain embodiments, the prediction of the response status of the patient to the immuno-oncology therapeutic further uses one or more medical history features of the patient. The third neural network is a segmentation algorithm that identifies a tumor region in the tissue sample, in specific embodiments. In some embodiments, the segmentation algorithm predicts one or more TIL mask structure features in the identified tumor region to generate the one or more TIL-related metrics. The predicted TIL mask structure feature is a predicted number of TIL clusters in the identified tumor region, a predicted size of TIL clusters in the identified tumor region, or a predicted TIL cluster spread in the identified tumor region. In some embodiments, the one or more TIL mask structure features comprises a total count of TILs in the identified tumor region, distance between TIL cells vs. distance between tumor cells, distance of TIL cells from tumor cells, average color of TIL cells, and/or average size of TIL cells. In some embodiments, the TIL % value is an intra-tumor TIL % (iTIL %), and the iTIL % is calculated as a percent of the identified tumor region in the tissue sample that is infiltrated by TIL. In certain embodiments, the TIL % value is a stromal TIL % (sTIL %), and the sTIL % value is calculated as a percent of stroma in the identified tumor region in the tissue sample that is infiltrated by TIL.

In particular embodiments, the first neural network model is a classification algorithm that generates a TMB indicator that is either a TMB positive classification or TMB negative classification for the tissue sample. In certain aspects, the classification algorithm generates the TMB positive classification if the tissue sample has a TMB value that exceeds a pre-set TMB threshold value, in specific embodiments. The first neural network model may be a regression algorithm that generates a TMB indicator that is a quantitative TMB score, in various embodiments, and the second neural network model is a classification algorithm that generates a PD-L1 expression level indicator that is either a PD-L1 expression positive classification or a PD-L1 expression level negative classification, in certain embodiments. The second neural network model is a classification algorithm that generates a PD-1 expression level indicator that is either a PD-1 expression positive classification or a PD-1 expression level negative classification, in specific embodiments. In some embodiments, the classification algorithm generates the PD-L1 expression positive classification if the tissue sample has a PD-L1 expression level value that excess a pre-set PD-L1 expression level value. The classification algorithm generates the PD-1 expression positive classification if the tissue sample has a PD-1 expression level value that excess a pre-set PD-1 expression level value, in specific embodiments. In some embodiments, the second neural network model is a regression algorithm that generates a PD-L1 expression level indicator that is a quantitative PD-L1 score. In various embodiments, the second neural network model is a regression algorithm that generates a PD-1 expression level indicator that is a quantitative PD-1 score. The quantitative PD-L1 score and/or PD-1 score is tumor proportion score (TPS), in some embodiments. The quantitative PD-L1 score and/or PD-1 score is a combined positive score (CPS), in some embodiments.

The immuno-oncology therapeutic is a check point inhibitor, such as a PD-L1 inhibitor or PD-1 inhibitor, in some embodiments.

In various embodiments, the method further comprises generating, by the one or more processors, via the third neural network model, one or more TIL masks from the one or more features, wherein the predicting of the response status of the patient to the immuno-oncology therapeutic further comprises using the one or more TIL masks.

In some embodiments, the tissue sample is from bladder and the patient response to the immuno-oncology therapeutic is related to bladder cancer. In various embodiments, wherein a determination is made that the patient is or will be a responder based on the response status; and wherein, in accordance with the determination, the patient is administered a therapeutically effective amount of the immuno-oncology therapeutic. In some embodiments, wherein a determination is made that the patient is not or will not be a responder based on the response status; and wherein, in accordance with the determination, the patient is not administered the immuno-oncology therapeutic. The tissue sample is from a patient known to have PD-L1-positive cancer or PD-1-positive cancer, in specific embodiments. The tissue sample is from a patient known to have any of the following: non-small cell lung cancer, melanoma, Hodgkin lymphoma, bladder cancer, kidney cancer, or breast cancer.

In particular embodiments, the immuno-oncology therapeutic targets PD-L1 or PD-1. The immuno-oncology therapeutic comprises one or more antibodies, adoptive cell therapies, immunomodulators, or a combination thereof, in specific embodiments, and the antibody may be a monoclonal antibody, a bispecific antibody, or a trispecific antibody. The adoptive cell therapy comprises immune cells that express one or more engineered antigen receptors, in specific embodiments. In some embodiments, the engineered antigen receptor is a chimeric antigen receptor, a non-natural T cell receptor, or a combination thereof. The adoptive cell therapy comprises T cells, natural killer cells, natural killer T cells, or a combination thereof, in various embodiments.

The method may further comprising obtaining the sample from the patient and/or diagnosing the patient as having cancer.

In specific embodiments, there is a method comprising: obtaining, by one or more processors from a data source, an image of a tissue sample from a patient and one or more features of the tissue sample; generating, by the one or more processors, via one or more neural network models one or more of the following: (a) one or more tumor mutation burden (TMB) indicators from the one or more features; (b) one or more programmed death-ligand 1 (PD-L1) expression level indicators from the one or more features or one or more programmed death-1 (PD-1) expression level indicators from the one or more features; and (c) one or more tumor infiltrating lymphocyte (TIL) percentage (%) values from the one or more features; predicting, by the one or more processors, via the one or more neural network models, a response status of the patient to an immuno-oncology therapeutic using one or more of: the one or more TMB indicators, the one or more PD-L1 expression level indicators, the one or more PD-1 expression level indicators, and/or the one or more TIL % values; and determining, based on the response status of the patient, whether to include the patient in a cohort of patients participating in a clinical trial.

In certain embodiments, there is a method comprising: obtaining, by one or more processors from a data source, an image of a tissue sample from a patient and one or more features of the tissue sample; and generating, by the one or more processors, via one or more neural network models a prediction of a response status of the patient to an immuno-oncology therapeutic using the image.

In particular embodiments, there is a system for predicting patient response to an immuno-oncology therapeutic, the system comprising: a data store for storing an image of a tissue sample from a patient, the image comprising one or more features of the tissue sample; a computing device communicatively connected to the data store, the computing device comprising: a first neural network model configured to generate one or more tumor mutation burden (TMB) indicators from the one or more features; a second neural network model configured to generate one or more programmed death-ligand 1 (PD-L1) expression level indicators from the one or more features and/or configured to generate one or more programmed death-1 (PD-1) expression level indicators from the one or more features; a third neural network model configured to calculate one or more tumor infiltrating lymphocyte (TIL)-related metrics from the one or more features; a fourth neural network model configured to calculate one or more cell-based features from the one or more features; and a fifth neural network model configured to predict a response status of the patient to the immuno-oncology therapeutic in accordance with the one or more TMB indicators, the one or more PD-L1 expression level indicators or the one or more PD-1 expression level indicators, respectively, the one or more TIL related metrics, and the one or more cell-based features; and a display system communicatively connected to the computing device and configured to display a result that indicates the response status of the patient.

In some embodiments, there is a method comprising: obtaining, by one or more processors from a data source, an image of a tissue sample from a patient and one or more features of the tissue sample; and training, by the one or more processors, one or more neural network models to predict a response status of the patient to an immuno-oncology therapeutic using the image.

In particular embodiments, there is a method for predicting patient response to an immuno-oncology therapeutic, the method comprising: obtaining, by one or more processors, an image of a tissue sample from a patient, the image comprising one or more features of the tissue sample; generating, by the one or more processors, one or more tumor mutation burden (TMB) indicators from the one or more features (optionally); generating, by the one or more processors, from the one or more features, one or more programmed death-ligand 1 (PD-L1) expression level indicators or one or more programmed death-1 (PD-1) expression level indicators; generating, by the one or more processors, one or more tumor infiltrating lymphocyte (TIL) related metrics from the one or more features; and training, by the one or more processors, a fourth neural network model to predict a response status of the patient to the immuno-oncology therapeutic based on one or more of: the one or more TMB indicators (when utilized), the one or more PD-L1 expression level indicators, the one or more PD-1 level indicators, and the one or more related metrics.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1A depicts a system for predicting a patient or treatment response to a therapeutic, in accordance with various embodiments.

FIG. 1B depicts another system for predicting a patient or treatment response to a therapeutic, in accordance with various embodiments.

FIG. 1C depicts another system for predicting a patient or treatment response to a therapeutic, in accordance with various embodiments

FIG. 2A illustrates a method for predicting a patient/treatment response to a therapeutic, in accordance with various embodiments.

FIG. 2B illustrates another method for predicting a patient/treatment response to a therapeutic, in accordance with various embodiments.

FIG. 2C illustrates another method for predicting a patient/treatment response to a therapeutic, in accordance with various embodiments.

FIG. 2D illustrates a method for predicting a patient/treatment response to a therapeutic, in accordance with various embodiments.

FIG. 2E illustrates another method for predicting a patient/treatment response to a therapeutic, in accordance with various embodiments.

FIG. 2F illustrates another method for predicting a patient/treatment response to a therapeutic, in accordance with various embodiments.

FIG. 3 is a block diagram of a computing system configured to perform a method of predicting a patient/treatment response to a therapeutic, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Embodiments of the disclosure include methods in which it is predicted whether or not an individual will respond to one or more certain therapies. In specific embodiments, the one or more certain therapies comprise one or more immuno-oncology therapeutics of any kind. In certain cases, the one or more immuno-oncology therapeutics target one or more checkpoint inhibitors. In particular embodiments, the one or more checkpoint inhibitors comprises PD-L1 or PD-1, or a mixture thereof. The type of immuno-oncology therapeutic may be of any kind, including a therapeutic that utilizes, at least in part, an antibody of any kind. In specific embodiments, the antibody comprises a monoclonal antibody and may or may not be humanized. The antibody may be utilized as an immuno-oncology therapeutic by itself, or the antibody may be configured as part of a chimeric molecule of any kind, such as an engineered antigen receptor. The antibody may be configured as part of a fusion protein of any kind.

In specific embodiments, an individual, such as a patient, is subject to methods in which multiple parameters are measured in order to determine if the individual will respond to a particular therapy. In particular cases, the parameters include one or more tumor mutation burden indicators, one or more PD-L1 expression level indicators and/or one or more PD-1 expression level indicators; and one or more TIL metric values. In some embodiments, patient characteristics, such as biological sex, age, individual or family health history (such as whether or not the individual and or one or more family members has had any disease including cancer before, including PD-L1-positive cancer and/or PD-1-positive cancer), and so forth, may also be included as a parameter or parameters to consider in order to determine if the individual will respond to a particular therapy.

In certain embodiments, an individual, such as a patient, is predicted to be or determined to be a responder to one or more immuno-oncology therapeutics. In some cases, an individual is predicted to be, or determined to be, at a higher probability of responding to one or more immuno-oncology therapeutics when compared to a particular population, such as the general population, a population of cancer patients, a population that lacks expression of one or more checkpoints, such as PD-L1 or PD-1, and so forth.

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

While the present teachings are described in conjunction with various embodiments, it is not intended that the present teachings be limited to such various embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### II. Systems and Workflows for Predicting Patient or Treatment Response to a Therapeutic

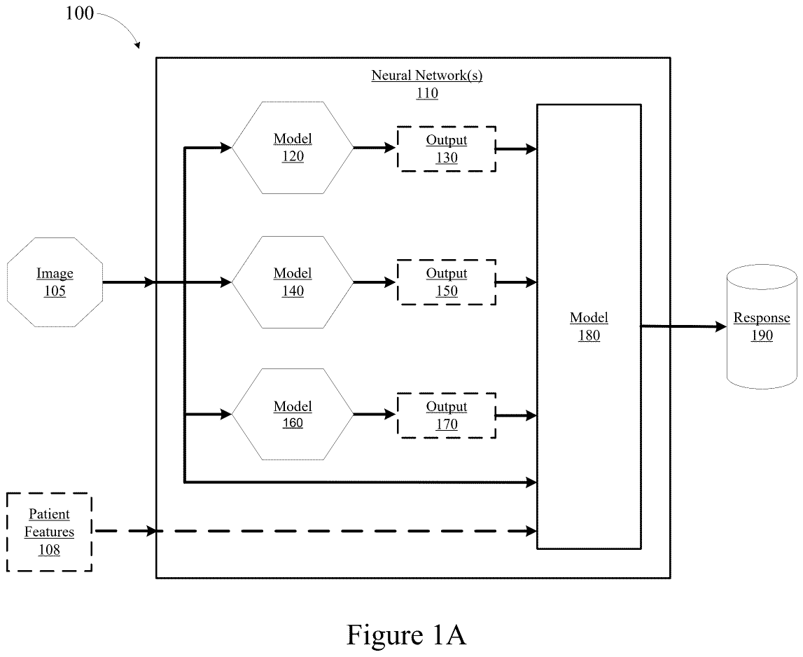

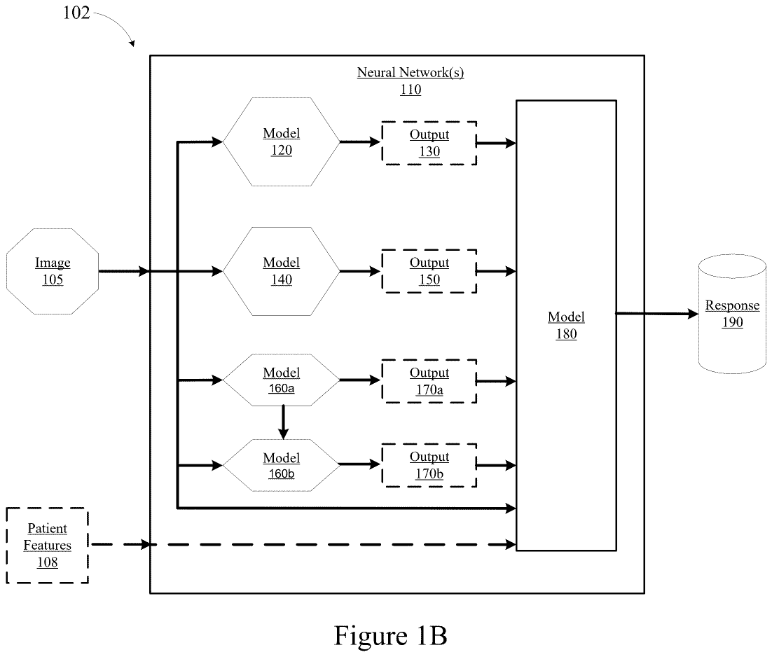

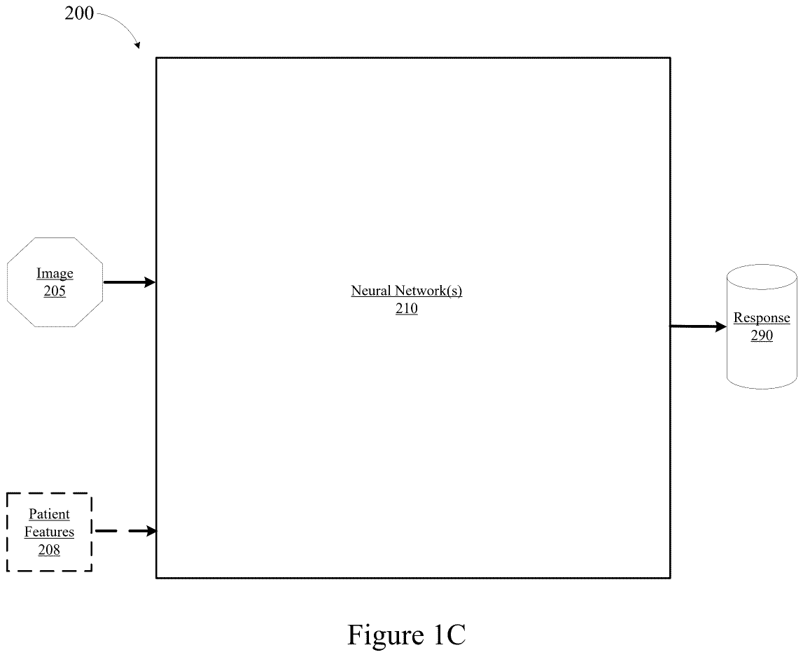

FIG. 1A depicts a system 100 for predicting a patient or treatment response to a therapeutic, in accordance with various embodiments. Although the system 100 may be applicable to any suitable therapeutics, such as for example, for cancers of any kind, the system 100 depicted in FIG. 1A is illustrated for predicting a patient response to an immuno-oncology therapeutic. As shown in FIG. 1A, the system 100 includes one or more neural networks 110 that can be used in analyzing an image 105 to make predictions of a patient response 190 (also referred to herein as “response 190”) based on the analysis of the image 105. In various embodiments, one or more patient features 108 may also be used in the analysis and prediction of the patient response 190 to the therapeutic.

In accordance with various embodiments, the image 105 can be an image of a tissue sample from the patient. In various embodiments, the image 105 can include one or more features of the tissue sample (e.g., shapes, sizes, distribution, and/or density of cells, etc.), which can be used in the analysis by the one or more neural networks 110 to predict the patient response 190 to the therapeutic. In various embodiments, the image 105 can include a Hematoxylin and Eosin (H&E)-stained tissue sample or any other samples from a patient that can be used in routine histopathological techniques for diagnosing a disease, such as cancer, including bladder cancer. In various embodiments, the image 105 can be acquired from a lab or a clinical department, such as a histology department, prior to analysis, or can be stored in a data store, database, or data storage for retrieving prior to analysis. The image 105 may be of tissue that is fresh or that was frozen.

In accordance with various embodiments, the one or more patient features 108 can include any feature related and/or pertinent to the patient that can help with the analysis or the prediction of the patient response to a therapeutic. Such features 108 may include, for example, but not limited to, age, biological sex, weight, smoking history, one or more symptoms, previous lines of treatment, previous treatment plans, time since diagnosis, the number of previous diagnoses to the same disease or any other diseases that may affect related organs or tissues, patient family medical information, presence or absence of one or more biomarkers for the disease, or any anatomical portions of the patient. In various embodiments, the one or more patient features 108 may include one or more medical history features of the patient. In various embodiments, the one or more patient features 108 may be extracted from a patient health profile, for example, from an electronic health record system.

As illustrated in FIG. 1A, the system 100 includes one or more neural networks 110 for analysis of input images, such as image 105 and for predicting the patient response 190 based on the analysis. In various embodiments, the one or more neural networks 110 include a first neural network model 120 (also referred to herein as “model 120”), a second neural network model 140 (also referred to herein as “model 140”), and a third neural network model 160 (also referred to herein as “model 160”). Each of the models 120, 140, and/or 160 can include a machine learning algorithm that can input the image 105, along with the one or more features of the tissue sample, to generate respective outputs 130, 150, and/or 170, as illustrated in FIG. 1A.

In various embodiments, the one or more neural networks 110 includes a fourth neural network model 180 (also referred to herein as “model 180”), which can include a machine learning algorithm that can ingest one or more of the outputs 130, 150, and 170 as input into the model 180 to generate the patient response 190. In various embodiments, the model 180 can input the image 105 and/or the one or more features of the tissue sample, as illustrated in FIG. 1A. In various embodiments, the model 180 can also input the one or more patient features 108 as part of the analysis to generate the patient response 190, as illustrated in FIG. 1A.

In various embodiments, the one or more neural networks 110, and thus, by extension, the models 120, 140, 160, and 180, may include, for example, without limitation, at least one, or any combination, of a Convolutional Neural Network (CNN), a Feedforward Neural Network (FNN), a Recurrent Neural Network (RNN), a Modular Neural Network (MNN), a Residual Neural Network (ResNet), an Ordinary Differential Equations Neural Networks (neural-ODE), or another type of neural network. Each of the one or more neural networks 110, and thus, by extension, the models 120, 140, 160, and 180, can include one or more algorithms, one or more mathematical techniques, one or more machine learning algorithms, or a combination thereof.

In various embodiments, the one or more neural networks 110, and thus, by extension, the models 120, 140, 160, and 180, may be trained using large amounts of datasets. In various embodiments, the training of the one or more neural networks 110, and thus, by extension, the models 120, 140, 160, and 180, can be self-supervised learning (SSL) or in supervised fashion. In various embodiments, the models 120, 140, 160, and 180, may be pre-trained using SSL and subsequently trained for one or more specific tasks (e.g., various indicators or metrics listed below) via supervised learning. Accordingly, the datasets for training the models can include annotated images by clinicians/pathologists. In various embodiments, each of the trained models 120, 140, 160, and/or 180 can then be configured to parse the one or more features of the tissue sample from the image 105, for example, to extract necessary data, learn from it, and then respectively make a determination or prediction, such as the outputs 130, 150, 170, and/or the patient response 190.

In order to predict a patient response to an immuno-oncology therapeutic, for example, the model 120 can be configured to generate one or more tumor mutation burden (TMB) indicators as the output 130, based on the one or more features of the image 105, in accordance with various embodiments. Similarly, the model 140 can be configured to generate one or more programmed death-ligand 1 (PD-L1) expression level indicators from the one or more features or configured to generate one or more programmed death-1 (PD-1) expression level indicators as the output 150 from the one or more features of the image 105, in accordance with various embodiments. In various embodiments, the immuno-oncology therapeutic is a checkpoint inhibitor, wherein the checkpoint inhibitor can be a PD-L1 inhibitor or PD-1 inhibitor. Likewise, the model 160 can be configured to calculate one or more tumor infiltrating lymphocyte (TIL)-related metrics as the output 170 from the one or more features of the image 105, in accordance with various embodiments.

In various embodiments, the model 120 includes a classification algorithm or a regression algorithm, both of which are trained using the annotated dataset as described above. In order to generate the one or more TMB indicators as the output 130, the classification algorithm of the model 120, for example, can ingest one or more features for the tissue sample from the image 105 to generate the output 130 as either a TMB positive classification or TMB negative classification. In various embodiments, the TMB positive classification is generated as the output 130 when a TMB value exceeds a pre-set TMB threshold value. In various embodiments, the regression algorithm of the model 120 can generate a TMB value or a quantitative TMB score as the TMB indicator for the output 130.

In various embodiments, the model 140 includes a classification algorithm or a regression algorithm, both of which are trained using the annotated dataset as described above. To generate one or more PD-L1 expression level indicators or one or more PD-1 expression level indicators as the output 150, either the classification algorithm or the regression algorithm can be employed to perform the analysis. In various embodiments, the classification algorithm of the model 140 can generate a PD-L1 expression level indicator as the output 150 that is either a PD-L1 expression positive classification or a PD-L1 expression level negative classification. In various embodiments, the classification algorithm of the model 140 can generate the PD-L1 expression positive classification if the tissue sample of the image 105 has a PD-L1 expression level value that exceeds a pre-set PD-L1 expression level value. In various embodiments, the regression algorithm of the model 140 can generate one or more PD-L1 expression level indicators as the output 150 that includes a quantitative PD-L1 score. In various embodiments, the quantitative PD-L1 score can be tumor proportion score (TPS) or a combined positive score (CPS).

In various embodiments, the classification algorithm of the model 140 can generate a PD-1 expression level indicator as the output 150 that is either a PD-1 expression positive classification or a PD-1 expression level negative classification. In various embodiments, the classification algorithm of the model 140 can generate the PD-1 expression positive classification if the tissue sample of the image 105 has a PD-1 expression level value that exceeds a pre-set PD-1 expression level value. In various embodiments, the regression algorithm of the model 140 can generate the one or more PD-1 expression level indicators as the output 150 that includes a quantitative PD-1 score. In various embodiments, the quantitative PD-1 score can be tumor proportion score (TPS) or a combined positive score (CPS).

In various embodiments, the model 160 may include one or more segmentation algorithms, each of which can be configured to generate one or more TIL masks, one or more tumor masks, one or more stroma masks, one or more necrosis masks, or one or more cell nuclei masks as the output 170 from the one or more features of the image 105. In various embodiments, the model 160 may include a region segmentation algorithm. In various embodiments, the model 160 may include a cell segmentation algorithm. In various embodiments, the model 160 can calculate TIL-related metrics, such as for example, but not limited to, the TIL %, sTIL %, or the number of TIL clusters, etc., by using the one or more TIL masks, one or more tumor masks, one or more storm masks, one or more necrosis masks, or one or more cell nuclei masks as the output 170.

In various embodiments, the model 160 may include a segmentation algorithm, which can be trained using the annotated dataset as described above. In various embodiments, the segmentation algorithm may be employed to generate an identification of a tumor region in the tissue sample based on the one or more features of the image 105, in accordance with various embodiments. This identification may be used to further generate or calculate one or more TIL-related metrics as the output 170 from the one or more features of the image 105. In various embodiments, the segmentation algorithm may be referred to as a region segmentation algorithm or a TIL region segmentation algorithm. In various embodiments, the segmentation algorithm may be referred to as a tumor region segmentation algorithm. In various embodiments, the segmentation algorithm may be configured to predict a TIL mask, which is used in combination with a tumor mask to calculate one or more TIL-related metrics as the output 170. In various embodiments, the one or more TIL-related metrics may include a predicted number of TIL clusters in the identified tumor region as the output 170. In various embodiments, the one or more TIL-related metrics may include a predicted size of TIL clusters in the identified tumor region as the output 170. In various embodiments, the one or more TIL-related metrics may include a predicted TIL cluster spread in the identified tumor region as the output 170. In various embodiments, the one or more TIL-related metrics may include an intra-tumor TIL % (iTIL %) as the output 170. In various embodiments, the iTIL % may be calculated as a percent of the identified tumor region in the tissue sample that is infiltrated by TIL as the output 170. In various embodiments, the one or more TIL-related metrics may include a stromal TIL % (sTIL %) as the output 170. In various embodiments, the sTIL % value may be calculated as a percent of stroma in the identified tumor region in the tissue sample that is infiltrated by TIL as the output 170.

In various embodiments, the model 160 may include a segmentation algorithm for cell segmentation, which can be referred to as a cell segmentation algorithm. In various embodiments, the cell segmentation algorithm may input the image 105 and perform segmentation of the cells as the output 170. In various embodiments, the segmented cells may be labeled based on the region segmentation output. For example, the segmented cell is labeled as a TIL cell if the cell is in a TIL region. In various embodiments, the cell segmentation algorithm is configured to calculate one or more features related to the cells, also referred to herein as cell-based features, contained in the image 105 of the tissue sample. In various embodiments, the calculated cell-based features may include morphology, color (nuclear), color (extra-nuclear), texture (energy, correlation, contrast, homogeneity, dissimilarity, angular second moment), chromatin skeleton morphology and color, among many other features that can be extracted. In various embodiments, the cell segmentation algorithm of the model 160 may be configured to generate a number of TIL cells or average number of TIL cells as the output 170. In various embodiments, the cell segmentation algorithm of the model 160 may be configured to generate an average tumor cell size as the output 170. In various embodiments, the cell segmentation algorithm of the model 160 may be configured to generate an average TIL cell size as the output 170. In various embodiments, the cell segmentation algorithm of the model 160 may be configured to generate an average distance between TIL cells as the output 170. In various embodiments, the cell segmentation algorithm of the model 160 may be configured to generate an average color of TIL cells as the output 170. In various embodiments, the cell segmentation algorithm of the model 160 may be configured to generate an average distance between TIL cells vs tumor cells, etc, as the output 170. In various embodiments, the one or more TIL-related metrics may include a total count of TILs in the identified tumor region as the output 170. In various embodiments, the one or more TIL-related metrics may include distance between TIL cells vs. distance between tumor cells as the output 170. In various embodiments, the one or more TIL-related metrics may include a distance of TIL cells from tumor cells as the output 170. In various embodiments, the one or more TIL-related metrics may include average color of TIL cells as the output 170. In various embodiments, the one or more TIL-related metrics may include average size of TIL cells as the output 170.

In various embodiments, the model 180 can be configured to generate a response status, for example, of the patient or treatment response 190 to the immuno-oncology therapeutic by applying one or more of the outputs 130, 150, and 170 as input into the model 180. In various embodiments, the one or more TMB indicators of the output 130, the one or more PD-L1 expression level indicators or one or more PD-1 expression level indicators, respectively, of the output 150, and the one or more TIL-related metrics of the output 170 can be used in the analysis by the model 180 to predict a response status of the patient to the immuno-oncology therapeutic, in accordance with various embodiments. As described above, the model 180 can include a machine learning algorithm that is trained using the patient's immune-oncology treatment response data, wherein the response 190 can include annotated labels of responder, partial responder, or non-responder as the ground truth for training. In various embodiments, the model 180 can be configured such that each of the outputs 130, 150, and 170 are weighed against one another in the analysis by the machine learning algorithm of the model 180. In various embodiments, the different weights applied to each of the outputs 130, 150, and 170 can affect and/or significantly alter the output 190 of the patient response to the therapeutic, such as the immune-oncology therapeutic.

In various embodiments, the model 180 can be trained using a set of H&E images from patients with response data available for the desired treatment or therapeutic, such as the immune-oncology therapeutic.

For example, the model 180 can be trained using the following four patients.
Patient 1, H&E image, Responder
Patient 2, H&E image, Non-responder
Patient 3, H&E image, Responder
Patient 4, H&E image, Responder

For each of the patient, each of the models 120, 140, and 160 can be run to generate the following datasets from the image 105, the outputs 130 (TMB score), 150 (PD-L1 score), and 170 (TIL mask and/or TIL features), respectively:
Patient 1, H&E image, TMB score, PD-L1 score, TIL mask, TIL features, Responder
Patient 2, H&E image, TMB score, PD-L1 score, TIL mask, TIL features, Non-responder
Patient 3, H&E image, TMB score, PD-L1 score, TIL mask, TIL features, Responder
Patient 4, H&E image, TMB score, PD-L1 score, TIL mask, TIL features, Responder

Using the above datasets of inputs 130, 150, and 170, the model 180 can be trained as follows: “H&E image, TMB score, PD-L1 score, TIL masks, TIL features,” along with the ground truth or label as either “responder” or “non-responder”, or in some instances, as “partial responder”. Once the model 180 is trained, the model 180 can be used to generate the patient response 190 based on a single image similar to the image 105 and the outputs that are generated by the trained models 120, 140, and 160 using one or more features of the single image.

FIG. 1B depicts a system 102 for predicting a patient or treatment response to a therapeutic, in accordance with various embodiments. The system 102 is similar to the system 100 described with respect to FIG. 1A where the image 105, the neural networks 110 having the models 120 and 140 generating the outputs 130 and 150, respectively, and the model 180 having to ingest the outputs 130 and 150, and/or the image 105, and/or the patient features 108 in order to generate the response 190. The system 102 is different to the system 100 in that instead of having the model 160 generate the output 170 that can be ingested by the model 180 to generate the response 190 (as in the system 100), the system 102 includes two models 160a and 160b, which respectively generate outputs 170a and 170b, which can be ingested by the model 180 to generate the response 190. In other words, the models 160a and 160b represent two separate segmentation algorithms. In various embodiments, the model 160a may include a region/TIL segmentation algorithm. In various embodiments, the model 160b may include a cell segmentation algorithm.

In various embodiments, the region/TIL segmentation algorithm of the model 160a may be employed to generate an identification of a tumor region in the tissue sample based on the one or more features of the image 105. In various embodiments, the identification may be used to further generate or calculate one or more TIL-related metrics as the output 170a from the one or more features of the image 105. In various embodiments, the region/TIL segmentation algorithm of the model 160a may be configured to predict a TIL mask, which is used in combination with a tumor mask to calculate one or more TIL-related metrics as the output 170a. In various embodiments, the one or more TIL-related metrics may include a predicted number of TIL clusters in the identified tumor region as the output 170a. In various embodiments, the one or more TIL-related metrics may include a predicted size of TIL clusters in the identified tumor region as the output 170a. In various embodiments, the one or more TIL-related metrics may include a predicted TIL cluster spread in the identified tumor region as the output 170a. In various embodiments, the one or more TIL-related metrics may include an intra-tumor TIL % (iTIL %) as the output 170a. In various embodiments, the iTIL % may be calculated as a percent of the identified tumor region in the tissue sample that is infiltrated by TIL as the output 170a. In various embodiments, the one or more TIL-related metrics may include a stromal TIL % (sTIL %) as the output 170a. In various embodiments, the sTIL % value may be calculated as a percent of stroma in the identified tumor region in the tissue sample that is infiltrated by TIL as the output 170a.

In various embodiments, the cell segmentation algorithm of the model 160b may input the image 105 and perform segmentation of the cells as the output 170b. In various embodiments, the cell segmentation algorithm of the model 160b may input a region/TIL mask generated by the model 160a and perform segmentation of the cells as the output 170b. In various embodiments, the segmented cells may be labeled based on the region segmentation output. For example, the segmented cell is labeled as a TIL cell if the cell is in a TIL region. In various embodiments, the cell segmentation algorithm is configured to calculate one or more features related to the cells, also referred to herein as cell-based features, contained in the image 105 of the tissue sample. In various embodiments, the calculated cell-based features may include morphology, color (nuclear), color (extra-nuclear), texture (energy, correlation, contrast, homogeneity, dissimilarity, angular second moment), chromatin skeleton morphology and color, among many other features that can be extracted. In various embodiments, the cell segmentation algorithm of the model 160b may be configured to generate a number of TIL cells or average number of TIL cells as the output 170b. In various embodiments, the cell segmentation algorithm of the model 160b may be configured to generate an average tumor cell size as the output 170b. In various embodiments, the cell segmentation algorithm of the model 160b may be configured to generate an average TIL cell size as the output 170b. In various embodiments, the cell segmentation algorithm of the model 160b may be configured to generate an average distance between TIL cells as the output 170b. In various embodiments, the cell segmentation algorithm of the model 160b may be configured to generate an average color of TIL cells as the output 170b. In various embodiments, the cell segmentation algorithm of the model 160b may be configured to generate an average distance between TIL cells vs tumor cells, etc., as the output 170b. In various embodiments, the one or more TIL-related metrics may include a total count of TILs in the identified tumor region as the output 170b. In various embodiments, the one or more TIL-related metrics may include distance between TIL cells vs. distance between tumor cells as the output 170b. In various embodiments, the one or more TIL-related metrics may include a distance of TIL cells from tumor cells as the output 170b. In various embodiments, the one or more TIL-related metrics may include average color of TIL cells as the output 170b. In various embodiments, the one or more TIL-related metrics may include average size of TIL cells as the output 170b.

In various embodiments, the model 180 can be configured to generate a response status, for example, of the patient or treatment response 190 to the immuno-oncology therapeutic by applying one or more of the outputs 130, 150, 170a and 170b as input into the model 180. In various embodiments, the one or more TMB indicators of the output 130, the one or more PD-L1 expression level indicators or one or more PD-1 expression level indicators, respectively, of the output 150, and the one or more TIL-related metrics of the output 170 can be used in the analysis by the model 180 to predict a response status of the patient to the immuno-oncology therapeutic, in accordance with various embodiments. As described above, the model 180 can include a machine learning algorithm that is trained using the patient's immune-oncology treatment response data, wherein the response 190 can include annotated labels of responder, partial responder, or non-responder as the ground truth for training. In various embodiments, the model 180 can be configured such that each of the outputs 130, 150, 170a, and 170b are weighed against one another in the analysis by the machine learning algorithm of the model 180. In various embodiments, the different weights applied to each of the outputs 130, 150, 170a, and 170b can affect and/or significantly alter the output 190 of the patient response to the therapeutic, such as the immune-oncology therapeutic.

For each of the patient, each of the models 120, 140, 160a, and 160b can be run to generate the following datasets from the image 105, the outputs 130 (TMB score), 150 (PD-L1 score), 170a (TIL mask and/or TIL features), and 170b (cell-based features), respectively:
Patient 1, H&E image, TMB score, PD-L1 score, TIL mask, TIL features, cell-based features, Responder
Patient 2, H&E image, TMB score, PD-L1 score, TIL mask, TIL features, cell-based features, Non-responder
Patient 3, H&E image, TMB score, PD-L1 score, TIL mask, TIL features, cell-based features, Responder
Patient 4, H&E image, TMB score, PD-L1 score, TIL mask, TIL features, cell-based features, Responder

Using the above datasets of inputs 130, 150, 170a, and 170b, the model 180 can be trained as follows: “H&E image, TMB score, PD-L1 score, TIL masks, TIL features, cell-based features” along with the ground truth or label as either “responder” or “non-responder”, or in some instances, as “partial responder”. Once the model 180 is trained, the model 180 can be used to generate the patient response 190 based on a single image similar to the image 105 and the outputs that are generated by the trained models 120, 140, 160a, and 160b using one or more features of the single image.

FIG. 1C depicts another system 200 for predicting a patient or treatment response to a therapeutic, in accordance with various embodiments. Similar to the system 100 as described with respect to FIG. 1A, the system 200 may be applicable to any suitable therapeutics, such as for example, for any type of cancer. Although the system 200 is depicted in FIG. 1C for predicting a patient response to an immuno-oncology therapeutic, the system 200 is suitable for generating a patient response to any other therapeutics. As shown in FIG. 1C, the system 200 includes one or more neural networks 210 that can be used in analyzing an image 205 to make predictions of a patient response 290 (also referred to herein as “response 290”) based on the analysis of the image 205. In various embodiments, one or more patient features 208 may also be used in the analysis and prediction of the patient response 290 to the therapeutic. Since the image 205 and the one or more patient features 208 may be substantially similar or identical to the image 105 and the one or more patient features 108, respectively, described with respect to FIG. 1A, they will not be described in further detail.

As illustrated in FIG. 1C, the system 200 includes one or more neural networks 210 for analysis of input images, such as image 205 and for predicting the patient response 290 based on the analysis. In various embodiments, the one or more neural networks 210 include one or more classification algorithms, one or more regression algorithms, and/or one or more segmentation algorithms for analysis and generating one or more final outputs and intermediatory outputs. In various embodiments, the intermediatory outputs may be generated by one or more of the classification algorithms, regression algorithms, and/or segmentation algorithms, and then used as inputs in another one of the one or more of the classification algorithms, regression algorithms, and/or segmentation algorithms to generate the final output(s). Each of the classification algorithms, regression algorithms, and/or segmentation algorithms of the one or more neural networks 210 is a machine learning algorithm that can input the image 205, along with the one or more features of the tissue sample, to generate the response 290, as illustrated in FIG. 1C.

In various embodiments, the one or more neural networks 210 may include, for example, without limitation, at least one of a Convolutional Neural Network (CNN), a Feedforward Neural Network (FNN), a Recurrent Neural Network (RNN), a Modular Neural Network (MNN), a Residual Neural Network (ResNet), an Ordinary Differential Equations Neural Networks (neural-ODE), or another type of neural network, or any combination thereof.

In various embodiments, the one or more neural networks 210 may be trained using large amounts of datasets. In various embodiments, the training of the one or more neural networks 210 can be self-supervised learning (SSL) or in supervised fashion. Accordingly, the datasets for training the models can include annotated images by clinicians/pathologists. In various embodiments, each of the trained classification algorithms, regression algorithms, and/or segmentation algorithms of the one or more neural networks 210 can then be configured to parse the one or more features of the tissue sample from the image 105, for example, to extract necessary data, learn from it, and then respectively make a determination or prediction, such as the patient response 190.

In various embodiments with relation to FIGS. 1A, 1B, or 1C, one or more neural network models 110 or 210 can be configured to generate a prediction of a response status of the patient to an immuno-oncology therapeutic using the image 105 or image 205. In order to predict a patient response to an immuno-oncology therapeutic, for example, the one or more neural networks 210 can be configured to generate one or more tumor mutation burden (TMB) indicators based on the one or more features of the image 205, in accordance with various embodiments. In various embodiments, the one or more neural networks 210 can be configured to generate one or more programmed death-ligand 1 (PD-L1) expression level indicators from the one or more features or configured to generate one or more programmed death-1 (PD-1) expression level indicators from the one or more features of the image 205, in accordance with various embodiments. In various embodiments, the one or more neural networks 210 can be configured to calculate one or more tumor infiltrating lymphocyte (TIL) related metrics from the one or more features of the image 205, in accordance with various embodiments.

In various embodiments, the one or more neural networks 210 are trained using the annotated dataset as described above. A classification algorithm of the one or more neural networks 210, for example, can ingest one or more features for the tissue sample from the image 205 to generate a TMB positive classification or TMB negative classification. These features may be used order to generate the one or more TMB indicators. In various embodiments, the TMB positive classification is generated as an intermediatory output when a TMB value exceeds a pre-set TMB threshold value. In various embodiments, the regression algorithm of the one or more neural networks 210 can generate a TMB value or a quantitative TMB score as the TMB indicator for the output 130.

In various embodiments, the one or more neural networks 210 can generate one or more PD-L1 expression level indicators or one or more PD-1 expression level indicators. In various embodiments, the one or more neural networks 210 can include a classification algorithm that can generate a PD-L1 expression level indicator as the output 150 that is either a PD-L1 expression positive classification or a PD-L1 expression level negative classification. In various embodiments, the classification algorithm can generate the PD-L1 expression positive classification if the tissue sample of the image 105 has a PD-L1 expression level value that exceeds a pre-set PD-L1 expression level value. In various embodiments, the one or more neural networks 210 can include a regression algorithm to generate one or more PD-L1expression level indicators as the output 150 that includes a quantitative PD-L1 score. In various embodiments, the quantitative PD-L1 score can be tumor proportion score (TPS) or a combined positive score (CPS).

In various embodiments, the classification algorithm can generate a PD-1 expression level indicator as an intermediatory output that is either a PD-1 expression positive classification or a PD-1 expression level negative classification. In various embodiments, the classification algorithm can generate the PD-1 expression positive classification if the tissue sample of the image 205 has a PD-1 expression level value that exceeds a pre-set PD-1 expression level value. In various embodiments, the regression algorithm can generate the one or more PD-1 expression level indicators that include a quantitative PD-1 score. In various embodiments, the quantitative PD-1 score can be tumor proportion score (TPS) or a combined positive score (CPS).

In various embodiments, the one or more neural networks 210 can include a segmentation algorithm, which is trained using the annotated dataset as described above. The segmentation algorithm is employed to generate an identification of a tumor region in the tissue sample based on the one or more features of the image 205, in accordance with various embodiments. These metrics may be used to generate or calculate one or more TIL-related metrics from the one or more features of the image 205. In various embodiments, the segmentation algorithm uses one or more TIL mask structure features in the identified tumor region to generate one or more TIL-related metrics as an intermediatory output. In various embodiments, the one or more TIL mask structure features may include a predicted number of TIL clusters in the identified tumor region as an intermediatory output. In various embodiments, the one or more TIL mask structure features may include a predicted size of TIL clusters in the identified tumor region as an intermediatory output. In various embodiments, the one or more TIL mask structure features may include a predicted TIL cluster spread in the identified tumor region as an intermediatory output. In various embodiments, the one or more TIL mask structure features may include a total count of TILs in the identified tumor region as an intermediatory output. In various embodiments, the one or more TIL mask structure features may include distance between TIL cells vs. distance between tumor cells as an intermediatory output. In various embodiments, the one or more TIL mask structure features may include a distance of TIL cells from tumor cells as an intermediatory output. In various embodiments, the one or more TIL mask structure features may include average color of TIL cells as an intermediatory output. In various embodiments, the one or more TIL mask structure features may include average size of TIL cells as an intermediatory output. In various embodiments, the one or more TIL-related metrics may include an intra-tumor TIL % (iTIL %) as an intermediatory output. In various embodiments, the iTIL % may be calculated as a percent of the identified tumor region in the tissue sample that is infiltrated by TIL an intermediatory output. In various embodiments, the one or more TIL-related metrics may include a stromal TIL % (sTIL %) as an intermediatory output. In various embodiments, the sTIL % value may be calculated as a percent of stroma in the identified tumor region in the tissue sample that is infiltrated by TIL as an intermediatory output.

In various embodiments, the one or more neural networks 210 may include one or more segmentation algorithms, each of which can be configured to generate one or more TIL masks, one or more tumor masks, one or more storm masks, one or more necrosis masks, or one or more cell nuclei masks as intermediatory outputs. In various embodiments, the one or more segmentation algorithms of the one or more neural networks 210 can calculate TIL-related metrics, such as for example, but not limited to, the TIL %, sTIL %, or the number of TIL clusters, etc., by using the one or more TIL masks, one or more tumor masks, one or more storm masks, one or more necrosis masks, or one or more cell nuclei masks.

In various embodiments, the one or more neural networks 210 can be configured to generate a response status, for example, of the patient or treatment response 290 to the immuno-oncology therapeutic by applying one or more of the intermediatory outputs described above as input into the one or more neural networks 210. In various embodiments, the one or more TMB indicators, the one or more PD-L1 expression level indicators or one or more PD-1 expression level indicators, and the one or more TIL-related metrics can be used in the analysis to predict a response status of the patient to the immuno-oncology therapeutic, in accordance with various embodiments. As described above, the one or more neural networks 210 can include a machine learning algorithm that is trained using the patient's immune-oncology treatment response data, wherein the response 290 can include annotated labels of responder, partial responder, or non-responder as the ground truth for training. In various embodiments, the one or more neural networks 210 can be configured such that each of the intermediatory outputs are weighed against one another in the analysis by the machine learning algorithms. In various embodiments, the different weights applied to various intermediatory outputs can affect and/or significantly alter the patient response 290 to the therapeutic, such as the immune-oncology therapeutic.

In various embodiments, no biomarkers are generated. In various embodiments, the one or more neural networks 210 directly predicts a patient's response from the image 205.

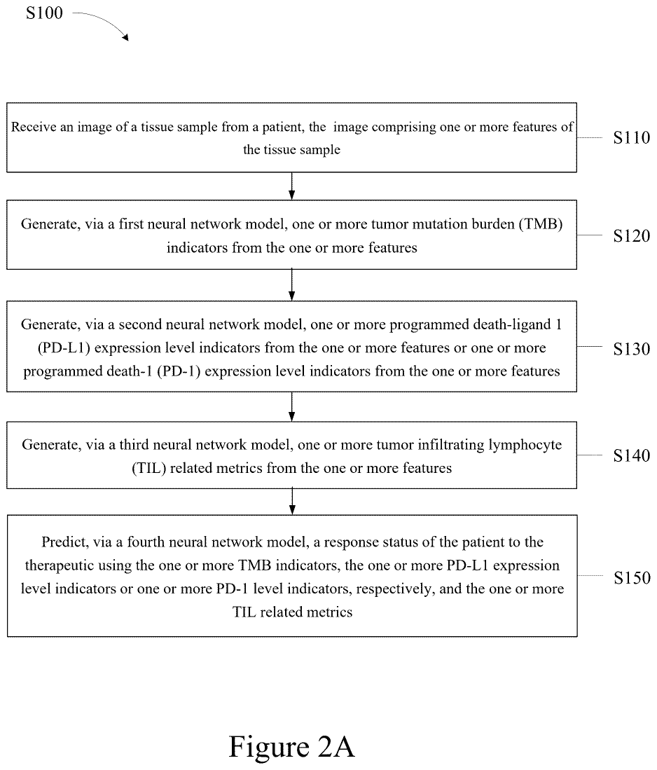

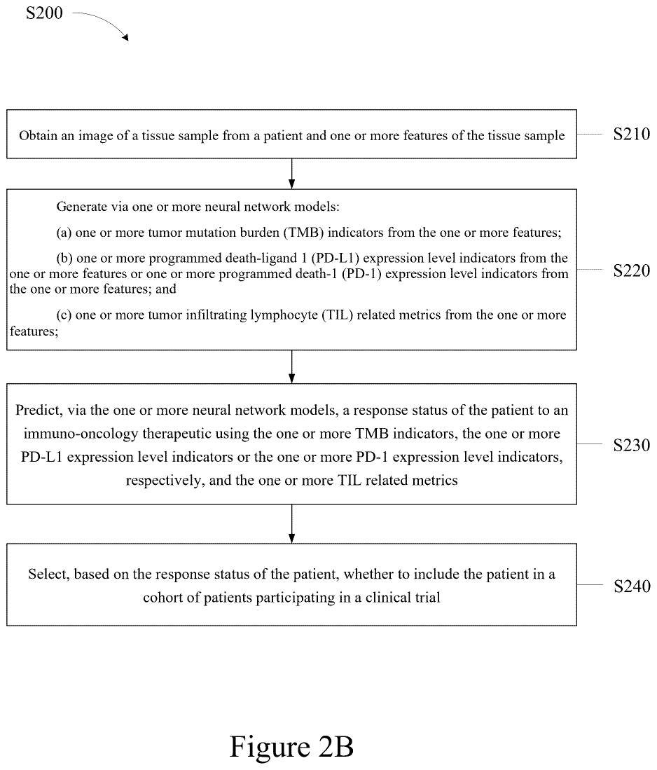

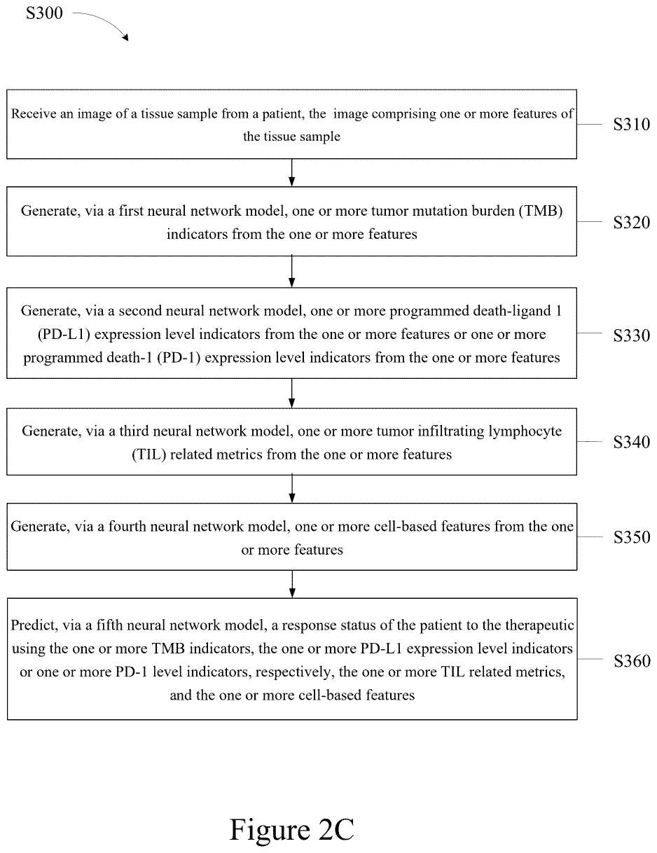

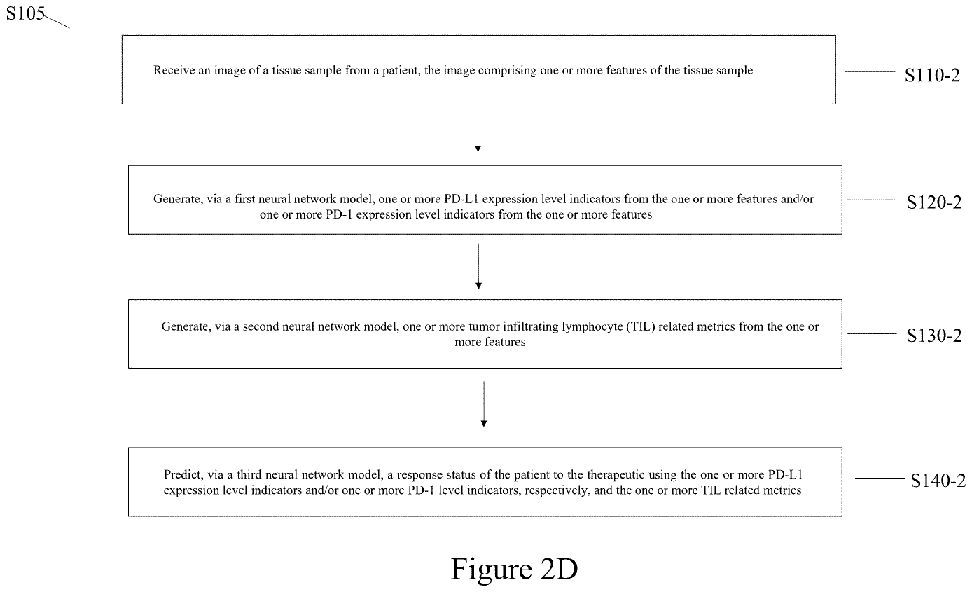

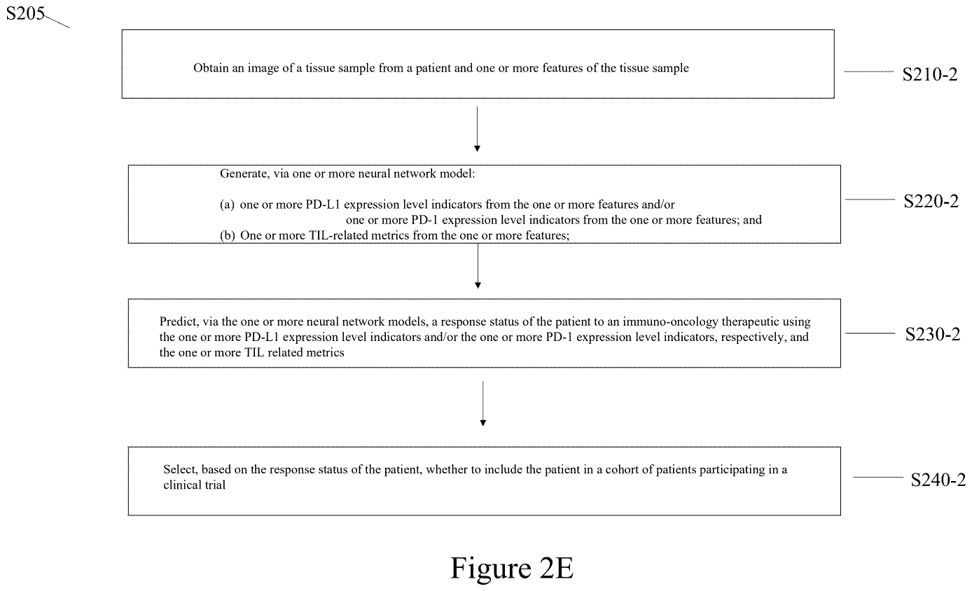

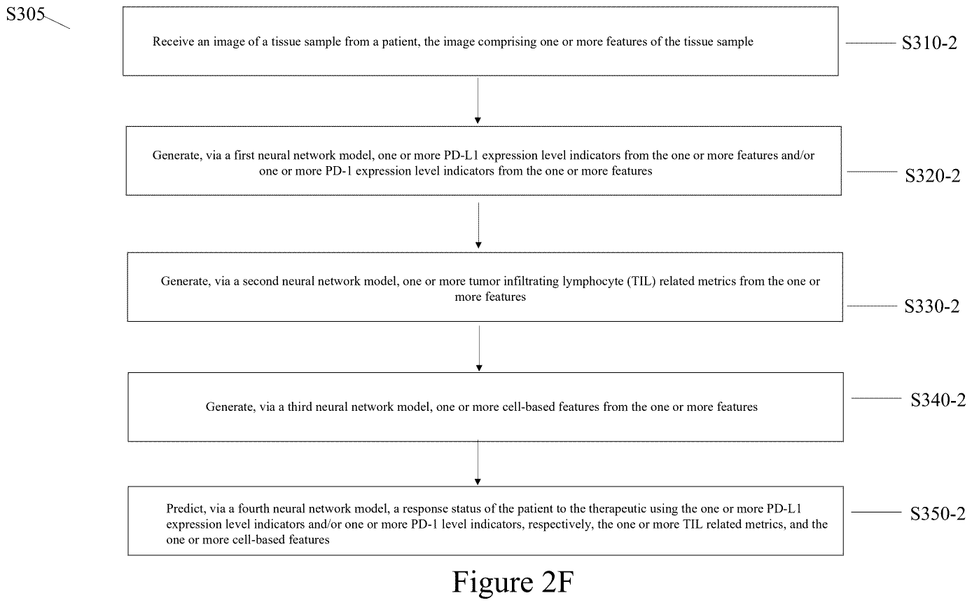

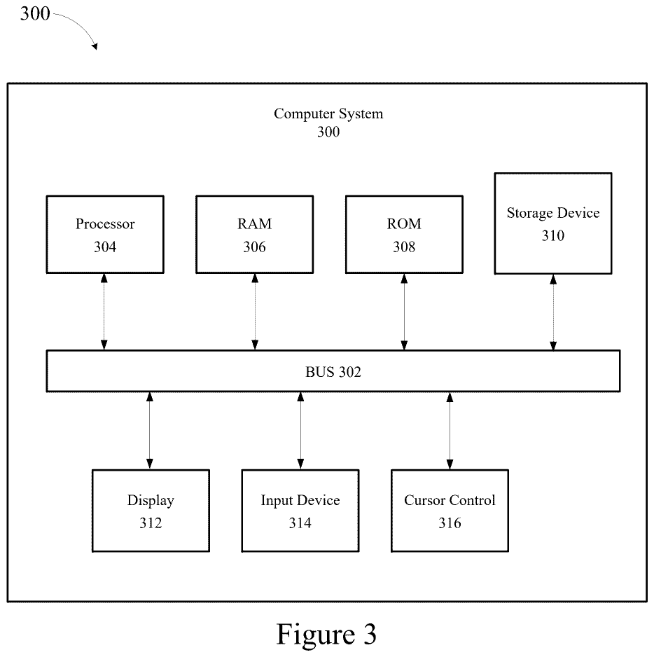

FIG. 2A illustrates a method S100 for predicting a patient/treatment response to a therapeutic, in accordance with various embodiments. The method S100 can be implemented for example, via a computing system 300 as described with respect to FIG. 3 below. As illustrated in FIG. 2A, the method S100 includes at step S110, obtaining or receiving, by one or more processors of the computing system 300, an image of a tissue sample, such as the image 105, from a patient; at step S120, generating, by the one or more processors, via a first neural network model, such as the model 120, one or more tumor mutation burden (TMB) indicators (e.g., as the output 130) from the one or more features; at step S130, generating, by the one or more processors, via a second neural network model, such as the model 140, one or more programmed death-ligand 1 (PD-L1) expression level indicators from the one or more features or one or more programmed death-1 (PD-1) expression level indicators (e.g., as the output 150) from the one or more features; at step S140, generating, by the one or more processors, via a third neural network model, such as the model 160, one or more tumor infiltrating lymphocyte (TIL)-related metrics (e.g., as the output 170) from the one or more features; and at step S150, predicting, by the one or more processors, via a fourth neural network model, such as the model 180, a response status of the patient, such as the response 190, to the immuno-oncology therapeutic using the one or more TMB indicators, the one or more PD-L1 expression level indicators or one or more PD-1 level indicators, respectively, and the one or more TIL-related metrics.

In various embodiments, the method S100 can optionally include displaying, on a display screen, a graphical representation indicating the response status of the patient. For instance, predicted response statuses of individuals may be displayed to a clinician or other health care provider, a clinical trial administrator, a researcher, or other entity via a graphical user interface, for example as part of a clinical trial management software or other software. In various embodiments, the method S100 can optionally include selecting, in accordance with the response status of the patient, whether to include the patient in a cohort of patients participating in a clinical trial. In specific embodiments, it may be desirable to determine whether or not a candidate for a clinical trial would be able to respond to the immuno-oncology therapeutic. Certain embodiments include treatment of an individual with a therapeutically effective amount of an immuno-oncology therapeutic wherein the individual is a responder as determined by systems or methods disclosed herein, and the individual may or may not be a participant in a clinical trial. In particular embodiments, an individual that is predicted to be a responder based on measurements, assaying, and/or generation of multiple neural network models as encompassed herein is administered a therapeutically effective amount of an immuno-oncology therapeutic.

In various embodiments of the method S100, the prediction of the response status of the patient to the immuno-oncology therapeutic further uses one or more medical history features of the patient. In various embodiments of the method S100, the third neural network is a segmentation algorithm that identifies a tumor region in the tissue sample. In various embodiments of the method S100, the segmentation algorithm predicts a TIL mask, which is used in combination with a tumor mask to calculate the one or more TIL-related metrics. In various embodiments of the method S100, the one or more TIL-related metrics includes a predicted number of TIL clusters in the identified tumor region. In various embodiments, the one or more TIL-related metrics includes a predicted size of TIL clusters in the identified tumor region.

In various embodiments of the method S100, the TIL-related metrics includes an intra-tumor TIL % (iTIL %). In various embodiments, the iTIL % is calculated as a percent of the identified tumor region in the tissue sample that is infiltrated by TIL. In various embodiments, the TIL % value is a stromal TIL % (sTIL %). In various embodiments, the sTIL % value is calculated as a percent of stroma in the identified tumor region in the tissue sample that is infiltrated by TIL.

In various embodiments of the method S100, the third neural network is a cell segmentation algorithm calculates one or more cell-based features. In various embodiments, the one or more cell-based features include morphology, color (nuclear), color (extra-nuclear), texture (energy, correlation, contrast, homogeneity, dissimilarity, angular second moment), or chromatin skeleton morphology and color. In various embodiments, the cell segmentation algorithm generates a number of TIL cells or average number of TIL cells. In various embodiments, the cell segmentation algorithm generates an average tumor cell size. In various embodiments, the cell segmentation algorithm generates an average TIL cell size. In various embodiments, the cell segmentation algorithm generates an average distance between TIL cells. In various embodiments, the cell segmentation algorithm generates an average color of TIL cells. In various embodiments, the cell segmentation algorithm generates an average distance between TIL cells vs tumor cells. In various embodiments, the one or more TIL-related metrics includes a predicted TIL cluster spread in the identified tumor region. In various embodiments, the one or more TIL-related metrics comprises a total count of TILs in the identified tumor region. In various embodiments, the one or more TIL-related metrics comprises distance between TIL cells vs. distance between tumor cells. In various embodiments, the one or more TIL-related metrics comprises distance of TIL cells from tumor cells. In various embodiments, the one or more TIL-related metrics comprises average color of TIL cells. In various embodiments, the one or more TIL-related metrics comprises average size of TIL cells.

In various embodiments of the method S100, the first neural network model is a classification algorithm that generates a TMB indicator that is either a TMB positive classification or TMB negative classification for the tissue sample. In various embodiments, the classification algorithm generates the TMB positive classification if the tissue sample has a TMB value that exceeds a pre-set TMB threshold value. In various embodiments, the first neural network model is a regression algorithm that generates a TMB indicator that is a quantitative TMB score.

In various embodiments of the method S100, the second neural network model is a classification algorithm that generates a PD-L1 expression level indicator that is either a PD-L1 expression positive classification or a PD-L1 expression level negative classification. In various embodiments, the second neural network model is a classification algorithm that generates a PD-1 expression level indicator that is either a PD-1 expression positive classification or a PD-1 expression level negative classification. In various embodiments, the classification algorithm generates the PD-L1 expression positive classification if the tissue sample has a PD-L1 expression level value that excess a pre-set PD-L1 expression level value. In various embodiments, the classification algorithm generates the PD-1 expression positive classification if the tissue sample has a PD-1 expression level value that excess a pre-set PD-1 expression level value. In various embodiments, the second neural network model is a regression algorithm that generates a PD-L1 expression level indicator that is a quantitative PD-L1 score. In various embodiments, the second neural network model is a regression algorithm that generates a PD-1 expression level indicator that is a quantitative PD-1 score. In various embodiments, the quantitative PD-L1 score is a tumor proportion score (TPS) or a combined positive score (CPS). In various embodiments, the quantitative PD-1 score is a tumor proportion score (TPS) or a combined positive score (CPS). In various embodiments, the immuno-oncology therapeutic is a check point inhibitor. In various embodiments, the check point inhibitor is a PD-L1 inhibitor or PD-1 inhibitor.

In various embodiments, the tissue sample is from bladder and the patient response to the immuno-oncology therapeutic is related to bladder cancer. In various embodiments, a determination is made that the patient is or will be a responder based on the response status. In various embodiments, in accordance with the determination, the patient is administered a therapeutically effective amount of the immuno-oncology therapeutic.

In various embodiments, a determination is made that the patient is not or will not be a responder based on the response status. In various embodiments, in accordance with the determination, the patient is not administered the immuno-oncology therapeutic.

In various embodiments, the tissue sample is from a patient known to have or suspected of having PD-L1-positive cancer or PD-1-positive cancer. In various embodiments, the tissue sample is from a patient known to have or suspected of having non-small cell lung cancer, melanoma, Hodgkin lymphoma, bladder cancer, kidney cancer, or breast cancer. In various embodiments, the immuno-oncology therapeutic targets PD-L1 or PD-1. In various embodiments, the immuno-oncology therapeutic comprises one or more antibodies, adoptive cell therapies, immunomodulators, or a combination thereof. In various embodiments, the antibody is a monoclonal antibody, a bispecific antibody, or a trispecific antibody. In various embodiments, the adoptive cell therapy comprises immune cells that express one or more engineered antigen receptors. In various embodiments, the engineered antigen receptor is a chimeric antigen receptor, a non-natural T cell receptor, or a combination thereof. In various embodiments, the adoptive cell therapy comprises T cells, natural killer cells, natural killer T cells, or a combination thereof. In various embodiments, the method S100 may optionally include the step of obtaining the sample from the patient. In various embodiments, the method S100 may include the step of diagnosing the patient as having cancer.

FIG. 2B illustrates another method S200 for predicting a patient/treatment response to a therapeutic, in accordance with various embodiments. The method S200 can be implemented for example, via a computing system 300 as described with respect to FIG. 3 below. As illustrated in FIG. 2B, the method S200 includes at step S210, obtaining or receiving, by one or more processors from a data source, an image of a tissue sample from a patient and one or more features of the tissue sample; at step S220, generating, by the one or more processors of the computing system 300, via one or more neural network models: (a) one or more tumor mutation burden (TMB) indicators from the one or more features; (b) one or more programmed death-ligand 1 (PD-L1) expression level indicators from the one or more features or one or more programmed death-1 (PD-1) expression level indicators from the one or more features; and (c) one or more tumor infiltrating lymphocyte (TIL) related metrics from the one or more features; at step S230, predicting, by the one or more processors, via the one or more neural network models, a response status of the patient to an immuno-oncology therapeutic using the one or more TMB indicators, the one or more PD-L1 expression level indicators or the one or more PD-1 expression level indicators, respectively, and the one or more TIL-related metrics; and at step S240, selecting, based on the response status of the patient, whether to include the patient in a cohort of patients participating in a clinical trial. In various embodiments, the method S200 can optionally include displaying, on a display screen, a graphical representation indicating the response status of the patient.

In alternate embodiments, step S220 can be omitted and step S230 may be directed to a prediction of a response status of the patient to the immune-oncology therapeutic from the image itself without the use of any calculated/generated biomarkers.

In various embodiments of the method S200, the prediction of the response status of the patient to the immuno-oncology therapeutic further uses one or more medical history features of the patient. In various embodiments of the method S200, the one or more neural network models can include a segmentation algorithm configured to predict a TIL mask, which is used in combination with a tumor mask to calculate one or more TIL-related metrics. In various embodiments of the method S200, the TIL-related metrics is a predicted number of TIL clusters in the identified tumor region. In various embodiments, the TIL-related metrics is a predicted size of TIL clusters in the identified tumor region.

In various embodiments of the method S200, the TIL-related metrics include an intra-tumor TIL % (iTIL %). In various embodiments, the iTIL % is calculated as a percent of the identified tumor region in the tissue sample that is infiltrated by TIL. In various embodiments, the TIL % value is a stromal TIL % (sTIL %). In various embodiments, the sTIL % value is calculated as a percent of stroma in the identified tumor region in the tissue sample that is infiltrated by TIL.

In various embodiments of the method S200, the one or more neural network models can include a segmentation algorithm for cell segmentation, which can be referred to as a cell segmentation algorithm. In various embodiments, the cell segmentation algorithm may input an image and perform segmentation of the cells. In various embodiments, the segmented cells may be labeled based on the region segmentation output. For example, the segmented cell is labeled as a TIL cell if the cell is in a TIL region. In various embodiments, the cell segmentation algorithm is configured to calculate one or more features related to the cells, also referred to herein as cell-based features, contained in the image. In various embodiments, the calculated cell-based features may include morphology, color (nuclear), color (extra-nuclear), texture (energy, correlation, contrast, homogeneity, dissimilarity, angular second moment), chromatin skeleton morphology and color, among many other features that can be extracted. In various embodiments, the cell segmentation algorithm may be configured to generate a number of TIL cells or average number of TIL cells. In various embodiments, the cell segmentation algorithm may be configured to generate an average tumor cell size. In various embodiments, the cell segmentation algorithm may be configured to generate an average TIL cell size. In various embodiments, the cell segmentation algorithm may be configured to generate an average distance between TIL cells. In various embodiments, the cell segmentation algorithm may be configured to generate an average color of TIL cells. In various embodiments, the cell segmentation algorithm may be configured to generate an average distance between TIL cells vs tumor cells. In various embodiments, the TIL-related metrics is a predicted TIL cluster spread in the identified tumor region. In various embodiments, the TIL-related metrics comprise a total count of TILs in the identified tumor region. In various embodiments, the TIL-related metrics can include a distance between TIL cells vs. distance between tumor cells. In various embodiments, the TIL-related metrics can include a distance of TIL cells from tumor cells. In various embodiments, the TIL-related metrics can include an average color of TIL cells. In various embodiments, the TIL-related metrics can include an average size of TIL cells.

In various embodiments of the method S200, the one or more neural network models can include a classification algorithm that generates a TMB indicator that is either a TMB positive classification or TMB negative classification for the tissue sample. In various embodiments, the classification algorithm generates the TMB positive classification if the tissue sample has a TMB value that exceeds a pre-set TMB threshold value.

In various embodiments, the one or more neural network models can include a regression algorithm that generates a TMB indicator that is a quantitative TMB score.

In various embodiments of the method S200, the one or more neural network models can include another classification algorithm that generates a PD-L1 expression level indicator that is either a PD-L1 expression positive classification or a PD-L1 expression level negative classification. In various embodiments, the classification algorithm generates a PD-1 expression level indicator that is either a PD-1 expression positive classification or a PD-1 expression level negative classification. In various embodiments, the classification algorithm generates the PD-L1 expression positive classification if the tissue sample has a PD-L1 expression level value that excess a pre-set PD-L1 expression level value. In various embodiments, the classification algorithm generates the PD-1 expression positive classification if the tissue sample has a PD-1 expression level value that excess a pre-set PD-1 expression level value.

In various embodiments, the one or more neural network models can include another regression algorithm that generates a PD-L1 expression level indicator that is a quantitative PD-L1 score. In various embodiments, the another regression algorithm generates a PD-1 expression level indicator that is a quantitative PD-1 score. In various embodiments, the quantitative PD-L1 score is a tumor proportion score (TPS) or a combined positive score (CPS). In various embodiments, the quantitative PD-1 score is a tumor proportion score (TPS) or a combined positive score (CPS). In various embodiments, the immuno-oncology therapeutic is a check point inhibitor. In various embodiments, the check point inhibitor is a PD-L1 inhibitor or PD-1 inhibitor.

In various embodiments, the tissue sample is from bladder and the patient response to the immuno-oncology therapeutic is related to bladder cancer. In various embodiments, a determination is made that the patient is or will be a responder based on the response status. In various embodiments, in accordance with the determination, the patient is administered a therapeutically effective amount of the immuno-oncology therapeutic.

In various embodiments, a determination is made that the patient is not or will not be a responder based on the response status. In various embodiments, in accordance with the determination, the patient is not administered the immuno-oncology therapeutic.

In various embodiments, the tissue sample is from a patient known to have or suspected of having PD-L1-positive cancer or PD-1-positive cancer. In various embodiments, the tissue sample is from a patient known to have or suspected of having non-small cell lung cancer, melanoma, Hodgkin lymphoma, bladder cancer, kidney cancer, or breast cancer. In various embodiments, the immuno-oncology therapeutic targets PD-L1 or PD-1. In various embodiments, the immuno-oncology therapeutic comprises one or more antibodies, adoptive cell therapies, immunomodulators, or a combination thereof. In various embodiments, the antibody is a monoclonal antibody, a bispecific antibody, or a trispecific antibody. In various embodiments, the adoptive cell therapy comprises immune cells that express one or more engineered antigen receptors. In various embodiments, the engineered antigen receptor is a chimeric antigen receptor, a non-natural T cell receptor, or a combination thereof. In various embodiments, the adoptive cell therapy comprises T cells, natural killer cells, natural killer T cells, or a combination thereof. In various embodiments, the method S100 may optionally include the step of obtaining the sample from the patient. In various embodiments, the method S100 may include the step of diagnosing the patient as having cancer.

FIG. 2C illustrates a method S300 for predicting a patient/treatment response to a therapeutic, in accordance with various embodiments. The method S300 can be implemented for example, via a computing system 300 as described with respect to FIG. 3 below. As illustrated in FIG. 2C, the method S300 includes at step S310, obtaining or receiving, by one or more processors of the computing system 300, an image of a tissue sample, such as the image 105, from a patient; at step S320, generating, by the one or more processors, via a first neural network model, such as the model 120, one or more tumor mutation burden (TMB) indicators (e.g., as the output 130) from the one or more features; at step S330, generating, by the one or more processors, via a second neural network model, such as the model 140, one or more programmed death-ligand 1 (PD-L1) expression level indicators from the one or more features or one or more programmed death-1 (PD-1) expression level indicators (e.g., as the output 150) from the one or more features; at step S340, generating, by the one or more processors, via a third neural network model, such as the model 160a, one or more tumor infiltrating lymphocyte (TIL)-related metrics (e.g., as the output 170a) from the one or more features; at step S350, generating, by the one or more processors, via a fourth neural network model, such as the model 160b, one or more cell-based features (e.g., as the output 170b) from the one or more features; and at step S360, predicting, by the one or more processors, via a fourth neural network model, such as the model 180, a response status of the patient, such as the response 190, to the immuno-oncology therapeutic using the one or more TMB indicators, the one or more PD-L1 expression level indicators or one or more PD-1 level indicators, respectively, the one or more TIL-related metrics, and the one or more cell-based features.

In any of FIGS. 2A-2C, the methods may lack the neural network model associated with one or more tumor mutation burden (TMB), as illustrated in FIGS. 2D-2F.

In FIG. 2D for method S105, S110-2 corresponds to S110 of FIG. 2A, In FIG. 2D, S120-2 corresponds to S130 of FIG. 2A, and in FIG. 2D, S130-2 corresponds to S140 of FIG. 2A. In FIG. 2D, S140-2 corresponds to S150 of FIG. 2A.

In FIG. 2E of method S205, S210-2 corresponds to S210 of FIG. 2B. In FIG. 2E, S220-2 corresponds to S220 of FIG. 2B (except S220-2 lacks a step related to TMB indicators). In FIG. 2E, S230-2 corresponds to S230 of FIG. 2B (except S230-2 lacks a step related to TMB indicators). In FIG. 2E, S240-2 corresponds to S240 of FIG. 2B.

In FIG. 2F of method 305, S310-2 corresponds to S310 of FIG. 2C. In FIG. 2F, S320-2 corresponds to S330 of FIG. 2C. In FIG. 2F, S330-2 corresponds to S340 of FIG. 2C. In FIG. 2F, S340-2 corresponds to S350 of FIG. 2C. In FIG. 2F, S350-2 corresponds to S360 of FIG. 2C (except S350-2 lacks a step related to TMB indicators.

### III. Computer Implemented System

FIG. 3 is a block diagram illustrating a computer system 300 configured to perform a method of predicting a patient/treatment response to a therapeutic, with which embodiments of the disclosed systems and methods, or portions thereof may be implemented, in accordance with various embodiments. For example, the illustrated computer system can be a local or remote computer system operatively connected to a control system for controlling or monitoring the systems and methods of the various embodiments herein. In various embodiments of the present teachings, computer system 300 can include a bus 302 or other communication mechanism for communicating information and a processor 304 coupled with bus 302 for processing information. In various embodiments, computer system 300 can also include a memory, which can be a random-access memory (RAM) 306 or other dynamic storage device, coupled to bus 302 for determining instructions to be executed by processor 304. Memory can also be used for storing temporary variables or other intermediate information during execution of instructions to be executed by processor 304. In various embodiments, computer system 300 can further include a read only memory (ROM) 308 or other static storage device coupled to bus 302 for storing static information and instructions for processor 304. A storage device 310, such as a magnetic disk or optical disk, can be provided and coupled to bus 302 for storing information and instructions.

In various embodiments, computer system 300 can be coupled via bus 302 to a display 312, such as a cathode ray tube (CRT) or liquid crystal display (LCD), for displaying information to a computer user. An input device 314, including alphanumeric and other keys, can be coupled to bus 302 for communication of information and command selections to processor 304. Another type of user input device is a cursor control 316, such as a mouse, a trackball or cursor direction keys for communicating direction information and command selections to processor 304 and for controlling cursor movement on display 312. This input device 314 typically has two degrees of freedom in two axes, a first axis (i.e., x) and a second axis (i.e., y), that allows the device to specify positions in a plane. However, it should be understood that input devices 314 allowing for 3-dimensional (x, y and z) cursor movement are also contemplated herein. In accordance with various embodiments, components 312/314/316, together or individually, can make up a control system that connects the remaining components of the computer system to the systems herein and methods conducted on such systems, and controls execution of the methods and operation of the associated system.

Consistent with certain implementations of the present teachings, results can be provided by computer system 300 in response to processor 304 executing one or more sequences of one or more instructions contained in memory 306. Such instructions can be read into memory 306 from another computer-readable medium or computer-readable storage medium, such as storage device 310. Execution of the sequences of instructions contained in memory 306 can cause processor 304 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 304 for execution. Such a medium can take many forms, including but not limited to, non-volatile media, volatile media, and transmission media. Examples of non-volatile media can include, but are not limited to, dynamic memory, such as memory 306. Examples of transmission media can include, but are not limited to, coaxial cables, copper wire, and fiber optics, including the wires that comprise bus 302.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 304 of computer system 300 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 300 as a standalone device or on a distributed network or shared computer processing resources such as a cloud computing network. In one or more embodiments, the computer system 300 may include a single computer (or computer system) or multiple computers in communication with each other in a distributed implementation.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 300, whereby processor 304 would execute the analyses and determinations provided by these engines, subject to instructions provided by any one of, or a combination of, memory components 306/308/310 and user input provided via input device 314.

While the present teachings are described in conjunction with various embodiments, it is not intended that the present teachings be limited to such various embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### V. Tumor Mutation Burden (TMB)

In certain embodiments, the systems, compositions, and methods of the disclosure utilize tumor mutation burden (TMB) as at least one parameter or metric to determine whether or not a patient will be a responder to a particular type of therapy. In various embodiments, the tumor mutation burden may include the total number of mutations (changes) present in the DNA of cancer cells. In specific cases, cancers (including solid tumors) that have a high number of mutations may be more likely to respond to certain types of immunotherapy, including immune-oncologic therapeutics. In specific embodiments, TMB is defined as the number of non-inherited mutations per million bases (Mb) of investigated genomic sequence (Merino, et al. (2020). J Immunother Cancer. 8 (1): e000147, which is incorporated by reference herein in its entirety). The measurement of TMB may occur by any suitable method, including by targeted panel sequencing, gene-targeted sequencing, or whole-exome sequencing, and in specific embodiments occurs by next generation sequencing (NGS), for example. The quantitative value of the TMB for a sample may be considered a TMB indicator, and in specific cases the quantity is measured as the number of mutations per Megabase (Mb). In the systems, compositions, and methods of the disclosure, one can generate (by one or more processors and via a neural network model) one or more tumor mutation burden (TMB) indicators from one or more features of an image of a tissue sample from an individual that has or is suspected of having bladder cancer. In specific embodiments, one can obtain, by one or more processors, an image of a tissue sample (from a patient) that comprises an image comprising one or more features of the tissue sample, followed by generating, by the one or more processors and via a first neural network model, one or more TMB indicators from the one or more features.

In particular embodiments, a neural network model is configured to generate one or more TMB indicators from one or more features of a patient tissue sample. In specific embodiments, the neural network model comprises a classification algorithm, and the one or more TMB indicators comprises a TMB indicator for the tissue sample that is either a TMB positive classification or TMB negative classification. In some embodiments, the neural network model comprises a classification algorithm, and the one or more TMB indicators comprises a TMB indicator for the tissue sample that is a positive classification if the tissue sample has a TMB value that exceeds a pre-set TMB threshold value. In such cases utilizing classification algorithm, in certain embodiments the output may be considered TMB High or TMB Low. In certain embodiments, TMB High may be >=10 while TMB Low is below 10.

However, in certain embodiments, the associated neural network model comprises a regression algorithm, and the one or more TMB indicators comprises a TMB indicator that is a quantitative TMB score. In specific cases, a TMB score may range from 0-100, and the pre-set TMB threshold value may be any value therein, including 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In particular cases, the TMB prediction parameter for the model is trained using hematoxylin and eosin (H&E) images, e.g., from muscle invasive bladder cancer (MIBC) patients. In various embodiments, in generation of the model and upon its use TMB is calculated from an NGS molecular panel applied to the tissue.

### VI. Checkpoint Expression Level

In embodiments of the disclosure, the systems, methods, and compositions utilize checkpoint expression level as at least one parameter or metric to determine whether or not a patient will be a responder to a particular type of therapy. Although in some cases the checkpoint is any kind of checkpoint, in specific embodiments the checkpoint is an inhibitory checkpoint. In certain embodiments, the checkpoint is Programmed Death-1 (PD-1), Programmed Death Ligand-1 (PD-L1). In particular embodiments, the expression of the checkpoint is one that is quantified based on images of tissue samples from a patient. In specific embodiments, the expression of the checkpoint is one that is quantified based on H&E images of tissue samples from a patient. In specific embodiments, a neural network model may be trained based on one or more features from H&E images (e.g., from bladder cancer patients, including muscle-invasive bladder cancer patients) with respect to one or more PD-L1 expression level indicators or PD-1 expression level indicators.

In certain embodiments, a neural network model comprises a classification algorithm and the one or more PD-L1 expression level indicators comprises a PD-L1 expression level indicator that is either a PD-L1 expression positive classification or a PD-L1 expression level negative classification. In other embodiments, a neural network model comprises a classification algorithm and the one or more PD-1 expression level indicators comprises a PD-1 expression level indicator that is either a PD-1 expression positive classification or a PD-1 expression level negative classification. In some embodiments, the classification algorithm generates the PD-L1 expression positive classification if the tissue sample has a PD-L1 expression level value that exceeds a pre-set PD-L1 expression level value. In such cases, an output may be PD-L1 High or PD-L1 Low. In some embodiments, the classification algorithm generates the PD-1 expression positive classification if the tissue sample has a PD-1 expression level value that exceeds a pre-set PD-1 expression level value. In such cases, an output may be PD-1 High or PD-1 Low. The threshold for PD-L1 High and PD-L1 Low, or PD-1 High and PD-1 Low, may be different depending on the immuno-oncology therapeutic and/or the cancer. In certain embodiments, there are different thresholds that are applied based on the drug and cancer.

In certain embodiments, a neural network model comprises a regression algorithm and the one or more PD-L1 expression level indicators comprises a quantitative PD-L1 score that may be a tumor proportion score (TPS) or a combined positive score (CPS). In certain embodiments, a neural network model comprises a regression algorithm and the one or more PD-1 expression level indicators comprises a quantitative PD-1 score that may be a tumor proportion score (TPS) or a combined positive score (CPS). For the regression algorithm for both PD-L1 and PD-1, the TPS score may range from 0-100, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. For the regression algorithm for both PD-L1 and PD-1, the CPS score may range from 0-100, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In specific embodiments, PD-L1 High or PD-1 High may be a TPS or a CPS≥1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In some embodiments, a model directly predicts the TPS and/or the CPS score (i.e. regression model). In some embodiments, a model directly predicts PD-L1 High vs. PD-L1 Low for different thresholds, or PD-1 High vs. PD-1 Low for different thresholds. In specific cases, one may utilize TPS>=1, TPS>=10, or TPS>=50, for example.

### VII. Tumor Infiltrating Lymphocyte (TIL)-Related Metrics

Tumor infiltrating lymphocytes are a type of white blood cell that has migrated from the blood towards a tumor. They can include both include T cells and B cells, and they recognize and kill cancer cells. In particular embodiments, the presence of lymphocytes in tumors is often associated with better clinical outcomes. The present disclosure provides systems, methods, and compositions that utilize TIL-related metrics as at least one parameter to determine whether or not a patient will be a responder to a particular type of therapy. The presence of TIL in a tumor sample is measured by any suitable method, including immunohistochemical staining, H&E staining, or both, in specific embodiments. In specific embodiments, the presence of TILs in the tumor is determined by a pathologist, and the pathologist may utilize IHC staining on tissue sample for visualization of TIL cells or they may be visualized in H&E slides. TILs have discerning characteristics to allow the pathologist to distinguish them from other cells, such as being more purple, darker, and rounder than tumor cells. In specific embodiments, TIL pathologist annotations on H&E images are utilized to train an algorithm. In specific embodiments, the pathologist assesses TILs in a stromal compartment of the tumor. In specific embodiments, the pathologist assesses TILs at the borders of an invasive tumor, for example. TILs outside of tumor borders, in zones of necrosis, in zones of fibrosis and/or associated with abscess are excluded, in specific cases. The pathologist may evaluate one or more H&E stained sections for the tumor.

In particular embodiments, a neural network model is configured to calculate one or more TIL-related metrics from one or more features of a tissue sample. In particular embodiments, one can calculate TIL-related metrics based on a TIL mask, tumor mask, necrosis mask, stroma masks, and cell nuclei segmentation mask. Several masks may be used to calculate “TIL-related” metrics (such as iTIL %, sTIL %, TIL clusters, etc.). The neural network may predict segmentation masks (i.e., a TIL mask on top of the tissue image, along with other masks such as tumor, stroma, necrosis, etc., and cell nuclei segmentations as well). These segmentation masks are then used to calculate metrics such as the following features: tumor infiltrating lymphocyte (TIL) percentage (%), total count of TILs, number of TIL clusters, distance between TIL cells vs. distance between tumor cells; distance of TIL cells from tumor cells; average color of TIL cells, average size of TIL cells, etc.

In various embodiments, the neural network model can be a cell segmentation algorithm that may input an image and perform segmentation of the cells. In various embodiments, the segmented cells may be labeled based on the region segmentation output. For example, the segmented cell is labeled as a TIL cell if the cell is in a TIL region. In various embodiments, the cell segmentation algorithm can calculate one or more cell-based features. In various embodiments, the cell-based features may include morphology, color (nuclear), color (extra-nuclear), texture (energy, correlation, contrast, homogeneity, dissimilarity, angular second moment), chromatin skeleton morphology and color, among many other features that can be extracted. In various embodiments, the cell segmentation algorithm may be configured to generate a number of TIL cells or average number of TIL cells, an average tumor cell size, an average TIL cell size, an average distance between TIL cells, an average color of TIL cells, an average distance between TIL cells vs tumor cells. In various embodiments, the TIL-related metrics is a predicted TIL cluster spread in the identified tumor region, a total count of TILs in the identified tumor region, va distance between TIL cells vs. distance between tumor cells, a distance of TIL cells from tumor cells, an average color of TIL cells, and/or an average size of TIL cells.

### VIII. Immuno-Oncology Therapeutics

The present disclosure provides embodiments wherein an individual in need of immune-oncology therapy is evaluated for being a responder to the immune-oncology therapy. Embodiments of the disclosure include predicting patient response to an immuno-oncology therapeutic. In specific embodiments, the immuno-oncology therapeutic is an immune check point inhibitor. The immuno-oncology therapeutic may be an antibody or may be adoptive cell therapy with immune cells, for example, or both may be utilized. In specific embodiments, the antibody is a monoclonal antibody, and the monoclonal antibody may target any checkpoint. In specific cases, the antibody may be monospecific or bispecific. The antibody may target PD-L1 or PD-1, for example. When adoptive cell therapy is utilized, the cells may be immune cells, such as T cells, natural killer (NK) cells, or NK T cells, as examples. In specific embodiments, the cells may be engineered to express a non-natural protein, such as an antigen receptor, and the antigen receptor may comprise an extracellular domain that is an antibody that targets an immune checkpoint. In other cases, the antigen receptor comprises an extracellular domain that binds an immune checkpoint. In specific embodiments, the immune-oncologic therapeutic is a check point inhibitor such as a PD-L1 inhibitor or PD-1 inhibitor. In particular embodiments, one may combine the immuno-oncology therapeutic with one or more other types of cancer therapies, such as surgery, radiation, hormone therapy, other immunotherapies, or chemotherapy.

In particular embodiments, methods comprise utilizing an image of a tissue sample from a patient, wherein the tissue sample comprises one or more features. In specific embodiments, the method comprises obtaining, by one or more processors from a data source, an image of a tissue sample from a patient and one or more features of the tissue sample. In either case, the method may comprise generating, such as by the one or more processors, via one or more neural network models: (a) one or more tumor mutation burden (TMB) indicators from the one or more features; (b) one or more programmed death-ligand 1 (PD-L1) expression level indicators from the one or more features or one or more programmed death-1 (PD-1) expression level indicators from the one or more features; and (c) one or more tumor infiltrating lymphocyte (TIL) metric values from the one or more features; predicting, by the one or more processors, via the one or more neural network models, a response status of the patient to an immuno-oncology therapeutic using the one or more TMB indicators, the one or more PD-L1 expression level indicators or the one or more PD-1 expression level indicators, respectively, and the one or more TIL metrics; and selecting, based on the response status of the patient, whether to include the patient in a cohort of patients participating in a clinical trial or whether to administer to the patient a therapeutically effective amount of one or more immune-oncology therapeutics.

In particular embodiments, the method comprises utilizing an image of a tissue sample from a patient, wherein the tissue sample comprises one or more features; obtaining, by one or more processors from a data source, an image of a tissue sample from a patient and one or more features of the tissue sample; generating, by the one or more processors, via one or more neural network models: (a) one or more tumor mutation burden (TMB) indicators from the one or more features; (b) one or more programmed death-ligand 1 (PD-L1) expression level indicators from the one or more features or one or more programmed death-1 (PD-1) expression level indicators from the one or more features; and (c) one or more tumor infiltrating lymphocyte (TIL) percentage (%) values (as an example of a TIL metric values) from the one or more features; predicting, by the one or more processors, via the one or more neural network models, a response status of the patient to an immuno-oncology therapeutic using the one or more TMB indicators, the one or more PD-L1 expression level indicators or the one or more PD-1 expression level indicators, respectively, and the one or more TIL % values; and selecting, based on the response status of the patient, whether to include the patient in a cohort of patients participating in a clinical trial.

Various embodiments of the method include obtaining, by one or more processors, an image of a tissue sample from a patient, the image comprising one or more features of the tissue sample. In some cases, the party that obtains the image is the same party that handles the processing and/or generating steps. In other cases, the party that obtains the image is not the same party that handles the processing and/or generating steps. The tissue sample may be obtained from storage or may be evaluated soon after collection. The tissue sample may or may not originate from a biopsy of an individual undergoing diagnosis of cancer. The individual may be known to have cancer or may be suspected of having cancer, based on one or more symptoms.

In specific embodiments, the systems, methods, and compositions of the disclosure may be utilized for any type of cancer, including hematological and solid tumors. The cancer may be of any stage or grade; it may or may not be refractory; it may or may not be metastatic. The cancer may be of the bladder, brain, breast, lung, colon, pancreas, prostate, liver, spleen, kidney, stomach, cervix, uterus, ovary, testes, gall bladder, bone, thyroid, skin, endometrium, rectum, and so forth.

In some embodiments, the subject has one or more symptoms of bladder cancer, such as hematuria; having to urinate more often than usual; pain or burning during urination; feeling a need to urinate right away, even when the bladder is not full; having trouble urinating or having a weak urine stream; having to get up to urinate many times during the night; being unable to urinate; lower back pain on one side; loss of appetite and weight loss; feeling tired or weak; swelling in the feet; bone pain; or a combination thereof. In such cases, the subject may be suspected of having bladder cancer or may be known to have bladder cancer. Diagnosis of bladder cancer can occur using cystoscopy, biopsy, blood tests, urine cytology, computerized tomography (CT) urogram, retrograde pyelogram, or a combination thereof. Other tests may be run to determine the extent of the cancer, such as Magnetic resonance imaging (MRI), Positron emission tomography (PET), Bone scan, and/or Chest X-ray. The bladder cancer may be urothelial carcinoma, squamous cell carcinoma, or adenocarcinoma. The bladder cancer may be noninvasive, non-muscle-invasive, or muscle-invasive. The bladder cancer may be of any stage or grade, including noninvasive papillary carcinoma, carcinoma in situ, T1, T2, T3, or T4.

Embodiments of the disclosure include methods of treatment for individuals determined to be responders based on systems, methods, and compositions encompassed herein. In specific embodiments, a responder individual is administered a therapeutically effective amount of one or more immune-oncology therapeutics based on measuring performed by systems, methods, and compositions encompassed herein. In specific embodiments, an individual that is not a responder is not administered one or more immune-oncology therapeutics based on measuring performed by systems, methods, and compositions encompassed herein. In various embodiments, a therapeutically effective amount of one or more immuno-oncology therapeutics is administered to an individual determined to have, or predicted to have, a response to one or more immuno-oncology therapeutics based on having the appropriate one or more TMB indicators, one or more PD-L1 and/or PD-1 expression level indicators, and one or more TIL-related metrics, all from one or more features of an image of a tissue sample. In particular embodiments, a therapeutically effective amount of one or more immuno-oncology therapeutics is administered to an individual determined to have, or predicted to have, a response to one or more immuno-oncology therapeutics based on the following: providing an image of a tissue sample from a patient, the tissue sample comprising and one or more features of the tissue sample; generating, by one or more processors, via a first neural network model, a one or more TMB indicators from the one or more features; generating, by the one or more processors, via a second neural network model, a one or more PD-L1 expression level indicators from the one or more features; generating, by the one or more processors, via a third neural network model, one or more TIL-related metrics from the one or more features; and predicting, by the one or more processors, via a fourth neural network model, that the individual will respond to the immuno-oncology therapeutic using the one or more TMB indicators, the one or more PD-L1 expression level indicators, and the one or more TIL-related metrics. In certain embodiments, a therapeutically effective amount of one or more immuno-oncology therapeutics is administered to an individual determined to have, or predicted to have, a response to one or more immuno-oncology therapeutics based on the following: obtaining, by one or more processors from a data source, an image of a tissue sample from a patient and one or more features of the tissue sample; generating, by the one or more processors, via one or more neural network models: (a) one or more tumor mutation burden (TMB) indicators from the one or more features; (b) one or more programmed death-ligand 1 (PD-L1) expression level indicators from the one or more features; and (c) one or more tumor infiltrating lymphocyte (TIL) percentage (%) values from the one or more features; determining or predicting, by the one or more processors, via the one or more neural network models, that the patient will respond to an immuno-oncology therapeutic using the one or more TMB indicators, the one or more PD-L1 expression level indicators, and the one or more TIL-related metric values.

### IX. Recitation of Embodiments

Embodiment 1. A system for predicting patient response to an immuno-oncology therapeutic, the system comprising: a data store for storing an image of a tissue sample from a patient, the image comprising one or more features of the tissue sample; a computing device communicatively connected to the data store, the computing device comprising: a first neural network model configured to generate one or more tumor mutation burden (TMB) indicators from the one or more features; a second neural network model configured to generate one or more programmed death-ligand 1 (PD-L1) expression level indicators from the one or more features or configured to generate one or more programmed death-1 (PD-1) expression level indicators from the one or more features; a third neural network model configured to calculate one or more tumor infiltrating lymphocyte (TIL)-related metrics from the one or more features; or a fourth neural network model configured to predict a response status of the patient to the immuno-oncology therapeutic in accordance with the one or more TMB indicators, the one or more PD-L1 expression level indicators or the one or more PD-1 expression level indicators, and/or the one or more TIL-related metrics; and a display system communicatively connected to the computing device and configured to display a result that indicates the response status of the patient.

Embodiment 2. The system of embodiment 1, wherein the prediction of the response status of the patient to the immuno-oncology therapeutic is performed in accordance with one or more medical history features of the patient.

Embodiment 3. The system of embodiment 1 or 2, wherein the third neural network model comprises a segmentation algorithm configured to generate an identification of a tumor region in the tissue sample based on the one or more features, wherein the identification is used to further generate the one or more TIL-related metrics.

Embodiment 4. The system of embodiment 3, wherein the segmentation algorithm is configured to predict a TIL mask, which is used in combination with a tumor mask to calculate one or more TIL related metrics.

Embodiment 5. The system of embodiment 4, wherein the one or more TIL-related metrics comprises a predicted number of TIL clusters in the identified tumor region.

Embodiment 6. The system of embodiment 4, wherein the one or more TIL-related metrics comprises a predicted size of TIL clusters in the identified tumor region.

Embodiment 7. The system of embodiment 4, wherein the one or more TIL-related metrics comprises a predicted TIL cluster spread in the identified tumor region.

Embodiment 8. The system of embodiment 4, wherein the one or more TIL-related metrics comprises a total count of TILs in the identified tumor region.

Embodiment 9. The system of embodiment 4, wherein the one or more TIL-related metrics comprises distance between TIL cells vs. distance between tumor cells

Embodiment 10. The system of embodiment 4, wherein the one or more TIL-related metrics comprises distance of TIL cells from tumor cells.

Embodiment 11. The system of embodiment 4, wherein the one or more TIL-related metrics comprises average color of TIL cells.

Embodiment 12. The system of embodiment 4, wherein the one or more TIL-related metrics comprises average size of TIL cells.

Embodiment 13. The system of any one of embodiments 1-12, wherein the one or more TIL-related metrics comprises an intra-tumor TIL % (iTIL %).

Embodiment 14. The system of embodiment 13, wherein the iTIL % is calculated as a percent of the identified tumor region in the tissue sample that is infiltrated by TIL.

Embodiment 15. The system of embodiment 1, wherein the one or more TIL-related metrics comprises a stromal TIL % (sTIL %).

Embodiment 16. The system of embodiment 15, wherein the sTIL % value is calculated as a percent of stroma in the identified tumor region in the tissue sample that is infiltrated by TIL.

Embodiment 17. The system of any one of embodiments 1-16, wherein the first neural network model comprises a classification algorithm and the one or more TMB indicators comprises a TMB indicator for the tissue sample that is either a TMB positive classification or TMB negative classification.

Embodiment 18. The system of embodiment 17, wherein the classification algorithm generates the TMB positive classification if the tissue sample has a TMB value that exceeds a pre-set TMB threshold value.

Embodiment 19. The system of any one of embodiments 1-16, wherein the first neural network model comprises a regression algorithm and the one or more TMB indicators comprises a TMB indicator that is a quantitative TMB score.

Embodiment 20. The system of any one of embodiments 1-19, wherein the second neural network model comprises a classification algorithm and the one or more PD-L1 expression level indicators comprises a PD-L1 expression level indicator that is either a PD-L1 expression positive classification or a PD-L1 expression level negative classification.

Embodiment 21. The system of any one of embodiments 1-19, wherein the second neural network model comprises a classification algorithm and the one or more PD-1 expression level indicators comprises a PD-1 expression level indicator that is either a PD-1 expression positive classification or a PD-1 expression level negative classification.

Embodiment 22. The system of embodiment 20, wherein the classification algorithm generates the PD-L1 expression positive classification if the tissue sample has a PD-L1 expression level value that exceeds a pre-set PD-L1 expression level value.

Embodiment 23. The system of embodiment 21, wherein the classification algorithm generates the PD-1 expression positive classification if the tissue sample has a PD-1 expression level value that exceeds a pre-set PD-1 expression level value.

Embodiment 24. The system of any one of embodiments 1-19, wherein the second neural network model comprises a regression algorithm and the one or more PD-L1 expression level indicators comprises a quantitative PD-L1 score.

Embodiment 25. The system of embodiment 1, wherein the second neural network model comprises a regression algorithm and the one or more PD-1 expression level indicators comprises a quantitative PD-1 score.

Embodiment 26. The system of embodiment 24, wherein the quantitative PD-L1 score is tumor proportion score (TPS).

Embodiment 27. The system of embodiment 24, wherein the quantitative PD-L1 score is a combined positive score (CPS).

Embodiment 28. The system of embodiment 25, wherein the quantitative PD-1 score is tumor proportion score (TPS).

Embodiment 29. The system of embodiment 25, wherein the quantitative PD-1 score is a combined positive score (CPS).

Embodiment 30. The system of any one of embodiments 1-29, wherein the immuno-oncology therapeutic is a check point inhibitor.

Embodiment 31. The system of embodiment 30, wherein the check point inhibitor is a PD-L1 inhibitor or PD-1 inhibitor.

Embodiment 32. The system of any one of embodiments 1-31, wherein the third neural network model is further configured to generate one or more TIL masks from the one or more features; and wherein the fourth neural network model is further configured to predict the response status of the patient to the immuno-oncology therapeutic using the one or more TIL masks.

Embodiment 33. The system of any one of embodiments 1, wherein the tissue sample is from the patient's bladder; and wherein the patient response to the immuno-oncology therapeutic is related to bladder cancer.

Embodiment 34. The system of any one of embodiments 1-33, wherein the tissue sample is from a patient known to have PD-L1-positive cancer.

Embodiment 35. The system of any one of embodiments 1-33, wherein the tissue sample is from a patient known to have PD-1-positive cancer.

Embodiment 36. The system of any one of embodiments 1-35, wherein the tissue sample is from a patient known to have any one or more of the following: non-small cell lung cancer, melanoma, Hodgkin lymphoma, bladder cancer, kidney cancer, and breast cancer.

Embodiment 37. The system of any one of embodiments 1-36, wherein the immuno-oncology therapeutic targets PD-L1 or PD-1.

Embodiment 38. The system of any one of embodiments 1-37, wherein the immuno-oncology therapeutic comprises one or more antibodies, one or more adoptive cell therapies, or a combination thereof.

Embodiment 39. The system of embodiment 38, wherein the one or more antibodies comprises at least one of: a monoclonal antibody, a bispecific antibody, or a trispecific antibody.

Embodiment 40. The system of embodiment 38, wherein the one or more adoptive cell therapies comprises immune cells that express one or more engineered antigen receptors.

Embodiment 41. The system of embodiment 40, wherein the one or more engineered antigen receptors comprises at least one of: a chimeric antigen receptor, a non-natural T cell receptor, or a combination thereof.

Embodiment 42. The system of any one of embodiments 38-41, wherein the one or more adoptive cell therapies comprises at least one of: T cells, natural killer cells, natural killer T cells, or a combination thereof.

Embodiment 43. The system of embodiment 1 or 2, wherein the third neural network model comprises a region/TIL segmentation algorithm and a cell segmentation algorithm.

Embodiment 44. The system of embodiment 43, wherein the cell segmentation algorithm calculates one or more cell-based features.

Embodiment 45. The system of embodiment 44, wherein the one or more cell-based features include morphology, color (nuclear), color (extra-nuclear), texture (energy, correlation, contrast, homogeneity, dissimilarity, or angular second moment).

Embodiment 46. The system of embodiment 43, wherein the cell segmentation algorithm generates a number of TIL cells or average number of TIL cells.

Embodiment 47. The system of embodiment 43, wherein the cell segmentation algorithm generates an average tumor cell size.

Embodiment 48. The system of embodiment 43, wherein the cell segmentation algorithm generates an average TIL cell size.

Embodiment 49. The system of embodiment 43, wherein the cell segmentation algorithm generates an average distance between TIL cells.

Embodiment 50. The system of embodiment 43, wherein the cell segmentation algorithm generates an average color of TIL cells.

Embodiment 51. The system of embodiment 43, wherein the cell segmentation algorithm generates an average distance between TIL cells vs tumor cells.

Embodiment 52. A method for predicting patient response to an immuno-oncology therapeutic, the method comprising: obtaining, by one or more processors, an image of a tissue sample from a patient, the image comprising one or more features of the tissue sample; generating, by the one or more processors, via a first neural network model, one or more tumor mutation burden (TMB) indicators from the one or more features; generating, by the one or more processors, via a second neural network model, one or more programmed death-ligand 1 (PD-L1) expression level indicators from the one or more features or one or more programmed death-1 (PD-1) expression level indicators from the one or more features; generating, by the one or more processors, via a third neural network model, one or more tumor infiltrating lymphocyte (TIL) related metrics from the one or more features; and predicting, by the one or more processors, via a fourth neural network model, a response status of the patient to the immuno-oncology therapeutic using the one or more TMB indicators, the one or more PD-L1 expression level indicators or the one or more PD-1 level indicators, and the one or more related metrics.

Embodiment 53. The method of embodiment 52, further comprising: selecting, in accordance with the response status of the patient, whether to include the patient in a cohort of patients participating in a clinical trial.

Embodiment 54. The method of embodiment 52 or 53, further comprising: displaying, on a display screen, a graphical representation indicating the response status of the patient.

Embodiment 55. The method of any one of embodiments 52-54, wherein the prediction of the response status of the patient to the immuno-oncology therapeutic further uses one or more medical history features of the patient.

Embodiment 56. The method of any one of embodiments 52-55, wherein the third neural network is a segmentation algorithm that identifies a tumor region in the tissue sample.

Embodiment 57. The method of embodiment 56, wherein the segmentation algorithm predicts one or more TIL mask structure features in the identified tumor region to generate the one or more TIL-related metrics.

Embodiment 58. The method of embodiment 57, wherein the predicted TIL mask structure feature is a predicted number of TIL clusters in the identified tumor region.

Embodiment 59. The method of embodiment 57, wherein the predicted TIL mask structure feature is a predicted size of TIL clusters in the identified tumor region.

Embodiment 60. The method of cla embodiment im 57, wherein the predicted TIL mask structure feature is a predicted TIL cluster spread in the identified tumor region.

Embodiment 61. The method of embodiment 57, wherein the one or more TIL mask structure features comprises a total count of TILs in the identified tumor region.

Embodiment 62. The method of embodiment 57, wherein the one or more TIL mask structure features comprises distance between TIL cells vs. distance between tumor cells

Embodiment 63. The method of embodiment 57, wherein the one or more TIL mask structure features comprises distance of TIL cells from tumor cells.

Embodiment 64. The method of embodiment 57, wherein the one or more TIL mask structure features comprises average color of TIL cells.

Embodiment 65. The method of embodiment 57, wherein the one or more TIL mask structure features comprises average size of TIL cells.

Embodiment 66. The method of any one of embodiments 52-65, wherein the TIL % value is an intra-tumor TIL % (iTIL %).

Embodiment 67. The method of embodiment 66, wherein the iTIL % is calculated as a percent of the identified tumor region in the tissue sample that is infiltrated by TIL.

Embodiment 68. The method of any one of embodiments 52-65, wherein the TIL % value is a stromal TIL % (sTIL %).

Embodiment 69. The method of embodiment 68, wherein the sTIL % value is calculated as a percent of stroma in the identified tumor region in the tissue sample that is infiltrated by TIL.

Embodiment 70. The method of any one of embodiments 52-69, wherein the first neural network model is a classification algorithm that generates a TMB indicator that is either a TMB positive classification or TMB negative classification for the tissue sample.

Embodiment 71. The method of embodiment 70, wherein the classification algorithm generates the TMB positive classification if the tissue sample has a TMB value that exceeds a pre-set TMB threshold value.

Embodiment 72. The method of any one of embodiments 52-69, wherein the first neural network model is a regression algorithm that generates a TMB indicator that is a quantitative TMB score.

Embodiment 73. The method of any one of embodiments 52-72, wherein the second neural network model is a classification algorithm that generates a PD-L1 expression level indicator that is either a PD-L1 expression positive classification or a PD-L1 expression level negative classification.

Embodiment 74. The method of any one of embodiments 52-72, wherein the second neural network model is a classification algorithm that generates a PD-1 expression level indicator that is either a PD-1 expression positive classification or a PD-1 expression level negative classification.

Embodiment 75. The method of embodiment 73, wherein the classification algorithm generates the PD-L1 expression positive classification if the tissue sample has a PD-L1 expression level value that excess a pre-set PD-L1 expression level value.

Embodiment 76. The method of embodiment 74, wherein the classification algorithm generates the PD-1 expression positive classification if the tissue sample has a PD-1 expression level value that excess a pre-set PD-1 expression level value.

Embodiment 77. The method of any one of embodiments 52-72, wherein the second neural network model is a regression algorithm that generates a PD-L1 expression level indicator that is a quantitative PD-L1 score.

Embodiment 78. The method of any one of embodiments 52-72, wherein the second neural network model is a regression algorithm that generates a PD-1 expression level indicator that is a quantitative PD-1 score.

Embodiment 79. The method of embodiment 77, wherein the quantitative PD-L1 score is tumor proportion score (TPS).

Embodiment 80. The method of embodiment 78, wherein the quantitative PD-1 score is tumor proportion score (TPS).

Embodiment 81. The method of embodiment 77, wherein the quantitative PD-L1 score is a combined positive score (CPS).

Embodiment 82. The method of embodiment 78, wherein the quantitative PD- 1 score is a combined positive score (CPS).

Embodiment 83. The method of any one of embodiments 52-82, wherein the immuno-oncology therapeutic is a check point inhibitor.

Embodiment 84. The method of embodiment 83, wherein the check point inhibitor is a PD-L1 inhibitor or PD-1 inhibitor.

Embodiment 85. The method of any one of embodiments 52-84, further comprising: generating, by the one or more processors, via the third neural network model, one or more TIL masks from the one or more features, wherein the predicting of the response status of the patient to the immuno-oncology therapeutic further comprises using the one or more TIL masks.

Embodiment 86. The method of any one of embodiments 52-85, wherein the tissue sample is from bladder and the patient response to the immuno-oncology therapeutic is related to bladder cancer.

Embodiment 87. The method of any one of embodiments 52-86, wherein a determination is made that the patient is or will be a responder based on the response status; and wherein, in accordance with the determination, the patient is administered a therapeutically effective amount of the immuno-oncology therapeutic.

Embodiment 88. The method of any one of embodiments 52-86, wherein a determination is made that the patient is not or will not be a responder based on the response status; and wherein, in accordance with the determination, the patient is not administered the immuno-oncology therapeutic.

Embodiment 89. The method of any one of embodiments 52-88, wherein the tissue sample is from a patient known to have PD-L1-positive cancer or PD-1-positive cancer.

Embodiment 90. The method of any one of embodiments 52-89, wherein the tissue sample is from a patient known to have any of the following: non-small cell lung cancer, melanoma, Hodgkin lymphoma, bladder cancer, kidney cancer, or breast cancer.

Embodiment 91. The method of any one of embodiments 52-90, wherein the immuno-oncology therapeutic targets PD-L1 or PD-1.

Embodiment 92. The method of any one of embodiments 52-91, wherein the immuno-oncology therapeutic comprises one or more antibodies, adoptive cell therapies, immunomodulators, or a combination thereof.

Embodiment 93. The method of embodiment 92, wherein the antibody is a monoclonal antibody, a bispecific antibody, or a trispecific antibody.

Embodiment 94. The method of embodiment 92, wherein the adoptive cell therapy comprises immune cells that express one or more engineered antigen receptors.

Embodiment 95. The method of embodiment 94, wherein the engineered antigen receptor is a chimeric antigen receptor, a non-natural T cell receptor, or a combination thereof.

Embodiment 96. The method of any one of embodiments 92-94, wherein the adoptive cell therapy comprises T cells, natural killer cells, natural killer T cells, or a combination thereof.

Embodiment 97. The method of any one of embodiments 52-96, further comprising: obtaining the sample from the patient.

Embodiment 98. The method of any one of embodiments 52-97, further comprising: diagnosing the patient as having cancer.

Embodiment 99. A method comprising: obtaining, by one or more processors from a data source, an image of a tissue sample from a patient and one or more features of the tissue sample; generating, by the one or more processors, via one or more neural network models one or more of the following: (a) one or more tumor mutation burden (TMB) indicators from the one or more features; (b) one or more programmed death-ligand 1 (PD-L1) expression level indicators from the one or more features or one or more programmed death-1 (PD-1) expression level indicators from the one or more features; and (c) one or more tumor infiltrating lymphocyte (TIL) percentage (%) values from the one or more features; predicting, by the one or more processors, via the one or more neural network models, a response status of the patient to an immuno-oncology therapeutic using one or more of: the one or more TMB indicators, the one or more PD-L1 expression level indicators, the one or more PD-1 expression level indicators, and/or the one or more TIL % values; and determining, based on the response status of the patient, whether to include the patient in a cohort of patients participating in a clinical trial.

Embodiment 100. A method comprising: obtaining, by one or more processors from a data source, an image of a tissue sample from a patient and one or more features of the tissue sample; and generating, by the one or more processors, via one or more neural network models a prediction of a response status of the patient to an immuno-oncology therapeutic using the image.

Embodiment 101. A system for predicting patient response to an immuno-oncology therapeutic, the system comprising: a data store for storing an image of a tissue sample from a patient, the image comprising one or more features of the tissue sample; a computing device communicatively connected to the data store, the computing device comprising: a first neural network model configured to generate one or more tumor mutation burden (TMB) indicators from the one or more features; a second neural network model configured to generate one or more programmed death-ligand 1 (PD-L1) expression level indicators from the one or more features or configured to generate one or more programmed death-1 (PD-1) expression level indicators from the one or more features; a third neural network model configured to calculate one or more tumor infiltrating lymphocyte (TIL)-related metrics from the one or more features; a fourth neural network model configured to calculate one or more cell-based features from the one or more features; and a fifth neural network model configured to predict a response status of the patient to the immuno-oncology therapeutic in accordance with the one or more TMB indicators, the one or more PD-L1 expression level indicators or the one or more PD-1 expression level indicators, respectively, the one or more TIL related metrics, and the one or more cell-based features; and a display system communicatively connected to the computing device and configured to display a result that indicates the response status of the patient.

Embodiment 102. A method comprising: obtaining, by one or more processors from a data source, an image of a tissue sample from a patient and one or more features of the tissue sample; and training, by the one or more processors, one or more neural network models to predict a response status of the patient to an immuno-oncology therapeutic using the image.

Embodiment 103. A method for predicting patient response to an immuno-oncology therapeutic, the method comprising: obtaining, by one or more processors, an image of a tissue sample from a patient, the image comprising one or more features of the tissue sample; generating, by the one or more processors, one or more tumor mutation burden (TMB) indicators from the one or more features; generating, by the one or more processors, from the one or more features, one or more programmed death-ligand 1 (PD-L1) expression level indicators or one or more programmed death-1 (PD-1) expression level indicators; generating, by the one or more processors, one or more tumor infiltrating lymphocyte (TIL) related metrics from the one or more features; and training, by the one or more processors, a fourth neural network model to predict a response status of the patient to the immuno-oncology therapeutic based on one or more of: the one or more TMB indicators, the one or more PD-L1 expression level indicators, the one or more PD-1 level indicators, and the one or more related metrics.

Embodiment 104. A method of treating a subject suffering from bladder cancer, the method comprising: administering to the subject an immuno-oncology therapeutic, wherein the subject has been determined to be responsive to the immuno-oncology therapeutic via a trained machine learning classifier that distinguishes between responsive and non-responsive subjects who have received the immuno-oncology therapeutic, based at least in part on analyzing in the subject of one or more of: a PD-L1 expression level, a PD-1 expression level, and one or more tumor infiltrating lymphocyte (TIL)-related metrics.

What is claimed is:

1. A system for predicting patient response to an immuno-oncology therapeutic, the system comprising:
a data store for storing an image of a tissue sample from a patient, the image comprising one or more features of the tissue sample;
a computing device communicatively connected to the data store, the computing device comprising:
a first neural network model configured to generate one or more programmed death-ligand 1 (PD-L1) expression level indicators from the one or more features and/or configured to generate one or more programmed death-1 (PD-1) expression level indicators from the one or more features;
a second neural network model configured to calculate one or more tumor infiltrating lymphocyte (TIL)-related metrics from the one or more features; or
a third neural network model configured to predict a response status of the patient to the immuno-oncology therapeutic in accordance with (a) the one or more PD-L1 expression level indicators and/or the one or more PD-1 expression level indicators and/or (b) the one or more TIL-related metrics; and
a display system communicatively connected to the computing device and configured to display a result that indicates the response status of the patient.

2. The system of claim 1, wherein the prediction of the response status of the patient to the immuno-oncology therapeutic is performed in accordance with one or more medical history features of the patient.

3. The system of claim 1, wherein the second neural network model comprises a segmentation algorithm configured to generate an identification of a tumor region in the tissue sample based on the one or more features, wherein the identification is used to further generate the one or more TIL-related metrics.

4. The system of claim 3, wherein the segmentation algorithm is configured to predict a TIL mask, which is used in combination with a tumor mask to calculate one or more TIL related metrics.

5. The system of claim 4, wherein the one or more TIL-related metrics comprises a predicted number of TIL clusters in the identified tumor region.

6. The system of claim 4, wherein the one or more TIL-related metrics comprises a predicted size of TIL clusters in the identified tumor region.

7. The system of claim 4, wherein the one or more TIL-related metrics comprises a predicted TIL cluster spread in the identified tumor region.

8. The system of claim 4, wherein the one or more TIL-related metrics comprises a total count of TILs in the identified tumor region.

9. The system of claim 4, wherein the one or more TIL-related metrics comprises distance between TIL cells vs. distance between tumor cells 10. The system of claim 4, wherein the one or more TIL-related metrics comprises distance of TIL cells from tumor cells.

11. The system of claim 4, wherein the one or more TIL-related metrics comprises average color of TIL cells.

12. The system of claim 4, wherein the one or more TIL-related metrics comprises average size of TIL cells.

13. The system of claim 1, wherein the one or more TIL-related metrics comprises an intra-tumor TIL % (iTIL %).

14. The system of claim 13, wherein the iTIL % is calculated as a percent of the identified tumor region in the tissue sample that is infiltrated by TIL.

15. The system of claim 1, wherein the one or more TIL-related metrics comprises a stromal TIL % (sTIL %).

16. The system of claim 15, wherein the sTIL % value is calculated as a percent of stroma in the identified tumor region in the tissue sample that is infiltrated by TIL.

17. The system of claim 1, further comprising:
a fourth neural network model configured to generate one or more tumor mutation burden (TMB) indicators from the one or more features,
wherein the third neural network model is further configured to predict a response status of the patient to the immune-oncology therapeutic in accordance with the one or more TMB indicators.

18. The system of claim 17, wherein the fourth neural network model comprises a classification algorithm and the one or more TMB indicators comprises a TMB indicator for the tissue sample that is either a TMB positive classification or TMB negative classification.

19. The system of claim 18, wherein the classification algorithm generates the TMB positive classification if the tissue sample has a TMB value that exceeds a pre-set TMB threshold value.

20. The system of claim 17, wherein the fourth neural network model comprises a regression algorithm and the one or more TMB indicators comprises a TMB indicator that is a quantitative TMB score.

21. The system of claim 1, wherein the first neural network model comprises a classification algorithm and the one or more PD-L1 expression level indicators comprises a PD-L1 expression level indicator that is either a PD-L1 expression positive classification or a PD-L1 expression level negative classification.

22. The system of claim 1, wherein the first neural network model comprises a classification algorithm and the one or more PD-1 expression level indicators comprises a PD-1 expression level indicator that is either a PD-1 expression positive classification or a PD-1 expression level negative classification.

23. The system of claim 22, wherein the classification algorithm generates the PD-L1 expression positive classification if the tissue sample has a PD-L1 expression level value that exceeds a pre-set PD-L1 expression level value.

24. The system of claim 22, wherein the classification algorithm generates the PD-1 expression positive classification if the tissue sample has a PD-1 expression level value that exceeds a pre-set PD-1 expression level value.

25. The system of claim 1, wherein the first neural network model comprises a regression algorithm and the one or more PD-L1 expression level indicators comprises a quantitative PD-L1 score.

26. The system of claim 1, wherein the first neural network model comprises a regression algorithm and the one or more PD-1 expression level indicators comprises a quantitative PD-1 score.

27. The system of claim 25, wherein the quantitative PD-L1 score is tumor proportion score (TPS).

28. The system of claim 25, wherein the quantitative PD-L1 score is a combined positive score (CPS).

29. The system of claim 26, wherein the quantitative PD-1 score is tumor proportion score (TPS).

30. The system of claim 26, wherein the quantitative PD-1 score is a combined positive score (CPS).

31. The system of claim 1, wherein the immuno-oncology therapeutic is a check point inhibitor.

32. The system of claim 31, wherein the check point inhibitor is a PD-L1 inhibitor or PD-1 inhibitor.

33. The system of claim 1, wherein the second neural network model is further configured to generate one or more TIL masks from the one or more features; and
wherein the third neural network model is further configured to predict the response status of the patient to the immuno-oncology therapeutic using the one or more TIL masks.

34. The system of claim 1, wherein the tissue sample is from the patient's bladder; and
wherein the patient response to the immuno-oncology therapeutic is related to bladder cancer.

35. The system of claim 1, wherein the tissue sample is from a patient known to have PD-L1-positive cancer.

36. The system of claim 1, wherein the tissue sample is from a patient known to have PD-1-positive cancer.

37. The system of claim 1, wherein the tissue sample is from a patient known to have any one or more of the following: non-small cell lung cancer, melanoma, Hodgkin lymphoma, bladder cancer, kidney cancer, and breast cancer.

38. The system of claim 1, wherein the immuno-oncology therapeutic targets PD-L1 or PD-1.

39. The system of claim 1, wherein the immuno-oncology therapeutic comprises one or more antibodies, one or more adoptive cell therapies, or a combination thereof.

40. The system of claim 39, wherein the one or more antibodies comprises at least one of:
a monoclonal antibody, a bispecific antibody, or a trispecific antibody.

41. The system of claim 39, wherein the one or more adoptive cell therapies comprises immune cells that express one or more engineered antigen receptors.

42. The system of claim 41, wherein the one or more engineered antigen receptors comprises at least one of: a chimeric antigen receptor, a non-natural T cell receptor, or a combination thereof.

43. The system of claim 39, wherein the one or more adoptive cell therapies comprises at least one of: T cells, natural killer cells, natural killer T cells, or a combination thereof.

44. The system of claim 1, wherein the second neural network model comprises a region/TIL segmentation algorithm and a cell segmentation algorithm.

45. The system of claim 44, wherein the cell segmentation algorithm calculates one or more cell-based features.

46. The system of claim 45, wherein the one or more cell-based features include morphology, color (nuclear), color (extra-nuclear), texture (energy, correlation, contrast, homogeneity, dissimilarity, or angular second moment).

47. The system of claim 44, wherein the cell segmentation algorithm generates a number of TIL cells or average number of TIL cells.

48. The system of claim 44, wherein the cell segmentation algorithm generates an average tumor cell size.

49. The system of claim 44, wherein the cell segmentation algorithm generates an average TIL cell size.

50. The system of claim 44, wherein the cell segmentation algorithm generates an average distance between TIL cells.

51. The system of claim 44, wherein the cell segmentation algorithm generates an average color of TIL cells.

52. The system of claim 44, wherein the cell segmentation algorithm generates an average distance between TIL cells vs tumor cells.

53. A method for predicting patient response to an immuno-oncology therapeutic, the method comprising:
obtaining, by one or more processors, an image of a tissue sample from a patient, the image comprising one or more features of the tissue sample;
generating, by the one or more processors, via a first neural network model, one or more programmed death-ligand 1 (PD-L1) expression level indicators from the one or more features and/or one or more programmed death-1 (PD-1) expression level indicators from the one or more features;
generating, by the one or more processors, via a second neural network model, one or more tumor infiltrating lymphocyte (TIL) related metrics from the one or more features; and
predicting, by the one or more processors, via a third neural network model, a response status of the patient to the immuno-oncology therapeutic using the one or more PD-L1 expression level indicators and/or the one or more PD-1 level indicators, and the one or more related metrics.

54. The method of claim 53, further comprising:
selecting, in accordance with the response status of the patient, whether to include the patient in a cohort of patients participating in a clinical trial.

55. The method of claim 53, further comprising:
displaying, on a display screen, a graphical representation indicating the response status of the patient.

56. The method of claim 53, wherein the prediction of the response status of the patient to the immuno-oncology therapeutic further uses one or more medical history features of the patient.

57. The method of claim 53, wherein the third neural network is a segmentation algorithm that identifies a tumor region in the tissue sample.

58. The method of claim 57, wherein the segmentation algorithm predicts one or more TIL mask structure features in the identified tumor region to generate the one or more TIL-related metrics.

59. The method of claim 58, wherein the predicted TIL mask structure feature is a predicted number of TIL clusters in the identified tumor region.

60. The method of claim 58, wherein the predicted TIL mask structure feature is a predicted size of TIL clusters in the identified tumor region.

61. The method of claim 58, wherein the predicted TIL mask structure feature is a predicted TIL cluster spread in the identified tumor region.

62. The method of claim 58, wherein the one or more TIL mask structure features comprises a total count of TILs in the identified tumor region.

63. The method of claim 58, wherein the one or more TIL mask structure features comprises distance between TIL cells vs. distance between tumor cells 64. The method of claim 58, wherein the one or more TIL mask structure features comprises distance of TIL cells from tumor cells.

65. The method of claim 58, wherein the one or more TIL mask structure features comprises average color of TIL cells.

66. The method of claim 58, wherein the one or more TIL mask structure features comprises average size of TIL cells.

67. The method of claim 53, wherein the TIL % value is an intra-tumor TIL % (iTIL %).

68. The method of claim 67, wherein the iTIL % is calculated as a percent of the identified tumor region in the tissue sample that is infiltrated by TIL.

69. The method of claim 53, wherein the TIL % value is a stromal TIL % (sTIL %).

70. The method of claim 69, wherein the sTIL % value is calculated as a percent of stroma in the identified tumor region in the tissue sample that is infiltrated by TIL.

71. The method of claim 53, further comprising:
generating, by the one or more processors, via a fourth neural network model, one or more tumor mutation burden (TMB) indicators from the one or more features,
wherein the predicting via the third neural network model is further performed using the one or more TMB indicators.

72. The method of claim 71, wherein the fourth neural network model is a classification algorithm that generates a TMB indicator that is either a TMB positive classification or TMB negative classification for the tissue sample.

73. The method of claim 72, wherein the classification algorithm generates the TMB positive classification if the tissue sample has a TMB value that exceeds a pre-set TMB threshold value.

74. The method of claim 71, wherein the fourth neural network model is a regression algorithm that generates a TMB indicator that is a quantitative TMB score.

75. The method of claim 53, wherein the first neural network model is a classification algorithm that generates a PD-L1 expression level indicator that is either a PD-L1 expression positive classification or a PD-L1 expression level negative classification.

76. The method of claim 53, wherein the first neural network model is a classification algorithm that generates a PD-1 expression level indicator that is either a PD-1 expression positive classification or a PD-1 expression level negative classification.

77. The method of claim 75, wherein the classification algorithm generates the PD-L1expression positive classification if the tissue sample has a PD-L1 expression level value that excess a pre-set PD-L1 expression level value.

78. The method of claim 76, wherein the classification algorithm generates the PD-1expression positive classification if the tissue sample has a PD-1 expression level value that excess a pre-set PD-1 expression level value.

79. The method of claim 53, wherein the first neural network model is a regression algorithm that generates a PD-L1 expression level indicator that is a quantitative PD-L1 score.

80. The method of claim 53, wherein the first neural network model is a regression algorithm that generates a PD-1 expression level indicator that is a quantitative PD-1 score.

81. The method of claim 79, wherein the quantitative PD-L1 score is tumor proportion score (TPS).

82. The method of claim 80, wherein the quantitative PD-1 score is tumor proportion score (TPS).

83. The method of claim 79, wherein the quantitative PD-L1 score is a combined positive score (CPS).

84. The method of claim 80, wherein the quantitative PD-1 score is a combined positive score (CPS).

85. The method of claim 53, wherein the immuno-oncology therapeutic is a check point inhibitor.

86. The method of claim 85, wherein the check point inhibitor is a PD-L1 inhibitor or PD-1 inhibitor.

87. The method of claim 53, further comprising:
generating, by the one or more processors, via the third neural network model, one or more TIL masks from the one or more features,
wherein the predicting of the response status of the patient to the immuno-oncology therapeutic further comprises using the one or more TIL masks.

88. The method of claim 53, wherein the tissue sample is from bladder and the patient response to the immuno-oncology therapeutic is related to bladder cancer.

89. The method of claim 53, wherein a determination is made that the patient is or will be a responder based on the response status; and
wherein, in accordance with the determination, the patient is administered a therapeutically effective amount of the immuno-oncology therapeutic.

90. The method of claim 53, wherein a determination is made that the patient is not or will not be a responder based on the response status; and
wherein, in accordance with the determination, the patient is not administered the immuno-oncology therapeutic.

91. The method of claim 53, wherein the tissue sample is from a patient known to have PD-L1-positive cancer or PD-1-positive cancer.

92. The method of claim 53, wherein the tissue sample is from a patient known to have any of the following: non-small cell lung cancer, melanoma, Hodgkin lymphoma, bladder cancer, kidney cancer, or breast cancer.

93. The method of claim 53, wherein the immuno-oncology therapeutic targets PD-L1 or PD-1.

94. The method of claim 53, wherein the immuno-oncology therapeutic comprises one or more antibodies, adoptive cell therapies, immunomodulators, or a combination thereof.

95. The method of claim 94, wherein the antibody is a monoclonal antibody, a bispecific antibody, or a trispecific antibody.

96. The method of claim 94, wherein the adoptive cell therapy comprises immune cells that express one or more engineered antigen receptors.

97. The method of claim 96, wherein the engineered antigen receptor is a chimeric antigen receptor, a non-natural T cell receptor, or a combination thereof.

98. The method of claim 94, wherein the adoptive cell therapy comprises T cells, natural killer cells, natural killer T cells, or a combination thereof.

99. The method of claim 53, further comprising:
obtaining the sample from the patient.

100. The method of claim 53, further comprising:
diagnosing the patient as having cancer.

101. A method comprising:
obtaining, by one or more processors from a data source, an image of a tissue sample from a patient and one or more features of the tissue sample;
generating, by the one or more processors, via one or more neural network models one or more of the following:
(a) one or more programmed death-ligand 1 (PD-L1) expression level indicators from the one or more features or one or more programmed death-1 (PD-1) expression level indicators from the one or more features; and
(b) one or more tumor infiltrating lymphocyte (TIL) percentage (%) values from the one or more features;
predicting, by the one or more processors, via the one or more neural network models, a response status of the patient to an immuno-oncology therapeutic using one or more of: the one or more PD-L1 expression level indicators, the one or more PD-1 expression level indicators, and/or the one or more TIL % values; and
determining, based on the response status of the patient, whether to include the patient in a cohort of patients participating in a clinical trial.

102. A method comprising:
obtaining, by one or more processors from a data source, an image of a tissue sample from a patient and one or more features of the tissue sample; and
generating, by the one or more processors, via one or more neural network models a prediction of a response status of the patient to an immuno-oncology therapeutic using the image.

103. A system for predicting patient response to an immuno-oncology therapeutic, the system comprising:
a data store for storing an image of a tissue sample from a patient, the image comprising one or more features of the tissue sample;
a computing device communicatively connected to the data store, the computing device comprising:
a first neural network model configured to generate one or more programmed death-ligand 1 (PD-L1) expression level indicators from the one or more features or configured to generate one or more programmed death-1 (PD-1) expression level indicators from the one or more features;
a second neural network model configured to calculate one or more tumor infiltrating lymphocyte (TIL)-related metrics from the one or more features;
a third neural network model configured to calculate one or more cell-based features from the one or more features; and
a fifth neural network model configured to predict a response status of the patient to the immuno-oncology therapeutic in accordance with the one or more PD-L1 expression level indicators or the one or more PD-1 expression level indicators, respectively, the one or more TIL related metrics, and the one or more cell-based features; and
a display system communicatively connected to the computing device and configured to display a result that indicates the response status of the patient.

104. A method comprising:
obtaining, by one or more processors from a data source, an image of a tissue sample from a patient and one or more features of the tissue sample; and
training, by the one or more processors, one or more neural network models to predict a response status of the patient to an immuno-oncology therapeutic using the image.

105. A method for predicting patient response to an immuno-oncology therapeutic, the method comprising:
obtaining, by one or more processors, an image of a tissue sample from a patient, the image comprising one or more features of the tissue sample;
generating, by the one or more processors, from the one or more features, one or more programmed death-ligand 1 (PD-L1) expression level indicators and/or one or more programmed death-1 (PD-1) expression level indicators;
generating, by the one or more processors, one or more tumor infiltrating lymphocyte (TIL) related metrics from the one or more features; and
training, by the one or more processors, a fourth neural network model to predict a response status of the patient to the immuno-oncology therapeutic based on one or more of:
the one or more PD-L1 expression level indicators, the one or more PD-1 level indicators, and the one or more related metrics.

106. A method of treating a subject suffering from bladder cancer, the method comprising:
administering to the subject an immuno-oncology therapeutic, wherein the subject has been determined to be responsive to the immuno-oncology therapeutic via a trained machine learning classifier that distinguishes between responsive and non-responsive subjects who have received the immuno-oncology therapeutic, based at least in part on analyzing in the subject of one or more of: a PD-L1 expression level, a PD-1 expression level, and one or more tumor infiltrating lymphocyte (TIL)-related metrics.
